SEQUENCE LISTING (5 sequences)

SEQ ID NO: 1 - protein, 375 aa, Lactobacillus reuteri F275
MTEQEQQTEELRCIGCGSIIQTEDPNGLGYTPKSALEKGKETGELYCQRCFRLRHYNEIAPVSLTDDDFLRLLNQIRDANALIVYVVDVFDFNGSLIPGLHRFVGDNPVLLVGNKEDLLPRSLRRPKLTDWIRQQANIAGLRPIDTVLVSAKKNHQIDHLLDVIEKYRHNRDVYVVGVTNVGKSTLINQIIKQRTGVKELITTSRFPGTTLDKIEIPLDDGHVLVDTPGIIHQEQMAHVLSPKDLKIVAPQKEIKPKTYQLNDGQTLFLGGVARFDYLHGERAGMVAYFDNNLPIHRTKLNNADNFYAKHLGDLLTPPTSDEKNEFPPLERYEFHITEKSDIVFEGLGWITVPAKTTVAAWVPKGVGALVRRAMI

SEQ ID NO: 2 - protein, 1221 aa, Lactobacillus reuteri 100-23
MKSRFFNKVDKFNGTFTQLEENSRRWEKLYRQRWAHDKVVRTTHGVNCTGSCSWNVYVKQGIITWEHQATDYPSCGPNIPGYEPRGCPRGASFSWYEYSPVRIKYPYIRGKLWELWTAAKKEHENPLDAWASIVEDPEKSKKYKKVRGHGGLIRVHRYEALEMISAACLYTIKKYGPDRIGGFTPIPAMSMMSFSAGARFIALMGGEQMSFYDWYADLPPASPQVWGEQTDVPESAEWYNSSYIIMWGSNVPLTRTPDAHFMTEVRYKGTKIVAVSPDYAENVKFADDWLAPEPGSDSAVAQAMTYVILDEFYQKHPVKRFIDYSKRFTDLPFMVELEPSTANDDHYTPGRFVRISDLVDDDTIVNPAWKTVVYDQNNHKIVVPNGTMGQEYNVKEKWNLELLDQNGNKIDPALSINDQGGETEQIIADFPAFSNDGNSVVQRHLPVKKLKFTDGQEHLVTNVYDLMMAQMGIDRTGNDDLAAKDAMDAESYFTPAWQESRSGVKAEQVIQIAREFAQNAAENEGRSMVIMGGGVNHWFNADMNYRNIINMLMLCGCVGMTGGGWAHYVGQEKLRPQEGWANITFANDWEKGGARQMQGTTWYYFATDQWRYEEIDNQAQKSPVWKSKHSYLHNADYNQMAIRLGWLPSYPQFDRNPLSFAKDYNTTDIDEISKKVVDELKKGTLHFAAEDPDANQNQPKAFFLWRSNLFASSGKGAEYFMKHLLGAENGLLAKPNDRVKPQDMIWRDKGAVGKLDLVVDMDFRMVSTPMYSDVVLPAATWYEKKDLSSTDMHPFIHPFNAAISPMWESKSDWQQFKLLAKTISEMAKKYMPGTFYDLKSAPLGHNTQGEIAQPYGKIKDWKNGETEPIPGKTMPSLKLVTRDYTKIYDKFITLGPNIVNNYGYKVDDQYDYLKGMNGTASEGIGAGCPLLDEDEKVCDAILRMSTASNGKLADRAWEKKQERTGEHLTDIGRGHADDSMSFKQITAQPQEAYPTPIGTSAKHGGARYTPFSLMTERNIPTFTLTGRQHFYIDHEIFREFGENMATYKPSLPPVVMAPGDVDVPPVKDEVTLKYMTPHGKWNIHTMYYDNLEMLTLFRGGPTIWISPQDADKIKVKDNDWIEVYNRNGVVTARAVVSVRMPEGSMYMYHAQDNEIYEPLSTITGNRGGSHNAPTQIHVKPTHMVGGYGQLSYGWNYYGPTGNQRDLYANVRKLRKVNWSED

SEQ ID NO: 3 - protein, 519 aa, Lactobacillus reuteri 100-23
MKIKAQISMVLNLDKCIGCHTCSVTCKNTWTNRPGAEYMWFNNVETKPGVGYPKRWEDEDQYHGGWTLNSKGKLKLRAGSKLNKIALGKIFYNNDMPELDNYYEPWTYDYKTLFGPEQKHQPVARPKSQITGEGMELTTGPNWDDDLAGSTEYVQQDPNMQKIEGDIKNNFEQAFMMYLPRLCEHCLNAPCVASCPSGAMYKRDEDGIVLVDQERCRGWRFCMTGCPYKKVYFNWKTHKAEKCTFCYPRIEEGQPTVCAETCVGRIRYIGAILYDADRVEEAASTPDESKLYEAQLGLFLDPNDPEVVKQALKDGISEEMIEAAQKSPIYKMAVKEKIAFPLHPEYRTMPMVWYIPPLSPVMSYFEGRDSIKNPEMIFPGIDQMRVPVQYLASLLTAGNVPVIKKALYKLAMMRLYMRAKTSGRDFDSSKLERVDLTEERATSLYRLLAIAKYEDRFVIPSSQKAEMEDAQTEQGSLGYDECEGCALAPQHKSMFKKAEAGKSTNQIYADSFYGGIWRD

SEQ ID NO: 4 - protein, 229 aa, Lactobacillus reuteri 100-23
MHNGWSIFLWVIYPYIMLASFFIGTFVRFKYFHPSITAKSSELFEKKWLMIGSITFHIGIILAFFGHCLGMFIPASWTAYFGITEHMYHIFGSLMMGIPAGILAFVGIAILTYRRMTCSRVYKTSDINDIIVDWALLVTIALGLACTITGAFIDYNYRTTISPWARSLFVLNPQWQLMRSVPLIYKIHVLCGLAIFGYFPYTRLVHALTLPWQYIFRRFIVYRRRARVY

SEQ ID NO: 5 - protein, 192 aa, Lactobacillus reuteri 100-23
MIDFRRLTDLKDTFAVLSRLIDYPDTETFSPEIRQLLLTDNALSTATRGELLSLFDELAALSSIEVQEMYAHLFEMNRRYTLYMSYYKMTDSRERGTILARLKMLYEMFGISEATSELSDYLPLLLEFLAYGDYTNDPRRQDIQLALSVIEDGTYTLLKNAVTDSDNPYIQLIRLTRSLIGSCIKMEVREDA

(19) United States
(12) Patent Application Publication
(10) Pub. No.: US 2011/0104240 A1
(43) Pub. Date: May 5, 2011

(54) Nitric Oxide Device and Method for Wound Healing, Treatment of Dermatological Disorders and Microbial Infections

(75) Inventors: Mitchell Lawrence Jones, Montreal (CA); Satya Prakash, Brossard (CA)

(73) Assignee: Micropharma Limited, Montreal (CA)

(21) Appl. No.: 13/000,555

(22) PCT Filed: Jun. 23, 2009
(86) PCT No.: PCT/CA09/00858

§ 371 Date: Dec. 21, 2010

Related U.S. Application Data
(60) Provisional application No. 61/075,040, filed on Jun. 24, 2008.
(60) Provisional application No. 61/097,978, filed on Sep. 18, 2008.
(60) Provisional application No. 61/153,696, filed on Feb. 19, 2009.
(60) Provisional application No. 61/166,430, filed on Apr. 3, 2009.

(51) Int. Cl. A61K 9/70; A61K 9/127; A61K 9/14; A61K 9/16; A61K 35/00; A61K 38/43; A61K 38/44; A61K 38/45; A61K 38/46; A61K 38/48; A61K 35/74; A61P 31/00; A61P 17/00; A61P 17/02; A61M 37/00

(52) U.S. Cl. 424/443; 424/450; 424/484; 424/490; 424/93.1; 424/93.4; 424/93.42; 424/93.45; 424/94.1; 424/94.4; 424/94.5; 424/94.6; 424/94.63; 424/94.64; 604/23

(57) ABSTRACT

The present disclosure provides a device having a casing with a barrier surface and a contact surface and a composition in the casing having a nitric oxide precursor and an isolated enzyme or live cell expressing an endogenous enzyme, for converting the nitric oxide gas precursor to nitric oxide gas or having activity on a substrate that produces a catalyst that causes the conversion of the nitric oxide gas precursor to nitric oxide gas. The present disclosure also provides methods and uses for treating wounds, microbial infections and dermatological disorders and for preserving meat products.

### FIELD OF THE DISCLOSURE

The present disclosure relates to methods, devices and compositions for the treatment of wounds, dermatological disorders and microbial infections with nitric oxide. In particular, the disclosure relates to methods, devices and compositions for topical administration of nitric oxide.

### BACKGROUND OF THE DISCLOSURE

Wound healing is a complicated process relying heavily on the integration of a multitude of control mechanisms, events, and factors. Inflammatory cells, keratinocytes, fibroblasts, and endothelial cells, as well as many enzymes and growth factors, must interact seamlessly for the normal healing process to occur (Blackytny et al. 2006). These factors will act together during the processes of clot formation, inflammation, re-epithelialisation, angiogenesis, granulation, contraction, scar formation, and tissue remodelling to ensure adequate wound healing. Several pathological conditions, including diabetes and venous stasis, are associated with a number of changes at the molecular level which ultimately disrupt normal wound healing and can lead to the formation of chronic wounds (Blackytny et al. 2006).

One of these changes is the pathological change in the regulation of nitric oxide (NO) during the wound healing process (Blackytny et al. 2006). Since the discovery in 1987 that endothelium derived relaxing factor (EDRF) is in fact NO, it has become evident that NO is a very widely distributed and multifunctional cellular messenger (Palmer et al. 1988). Normally, NO is produced by the enzyme nitric oxide synthase (NOS) from the amino acid L-arginine. NO is a transitory free radical that is responsible for the regulation of blood pressure and the control of platelet aggregation (Mollace et al. 1990), and may be involved in vascular injury caused by tissue deposition of immune complexes (Mulligan et al. 1991). During normal healing, the production of NO radical shows a very distinct time course with initially high concentrations which aid in inhibiting and clearing bacterial infection followed by lower levels of the free radical allowing for the normal wound healing processes to take place (Blackytny et al. 2006). It is believed that the body's natural response to injury is with initially high NO concentrations for reducing the bacterial count, removing dead cells, and promoting healing. After a few days of this preparation of the wound bed, the body produces a new low NO level to promote further healing (Stenzler et al. 2006). If a wound fails to heal, however, or becomes infected, the body maintains the circulating NO at a high level and the wound is then caught in a vicious cycle preventing it from healing (Stenzler et al. 2006).

Infected wounds pose a specific and significant problem to wound care specialists treating a chronic wound, non-healing ulcer, or healthy post surgical wound for that matter. Typically, these wounds have been cared for by nurses, internists, plastic surgeons, and infectious disease specialists who use daily wet-to-dry dressing changes for debridement and topical or systemic antibiotics for treatment of the infection. Systemic and topical antibiotics, as well as other topical anti-microbial agents such as colloidial silver polymyxins or dye compounds, however, have become increasingly less effective against common pathogens. A worldwide increase in drug resistant strains of bacteria since the introduction of antimicrobial agents has documented this well accepted trend. Both Gorwitz and Anstead et al have recently reviewed Methicillin-resistant Staphylococcus aureus (MRSA) infections in skin and soft tissue, describing its emergence as a common cause of infection in children and adults in both community and hospital settings (Anstead et al. 2007; Gorwitz 2008). Linares 2001 has recently reviewed the emergence of vancomycin intermediate resistant Staphylococcus aureus (VISA) and glycopeptide-intermediate S. aureus (GISA), for which few drugs and strategies to fight infection exist. Further, Nordmann et al. recently reviewed the new resistance problems that have emerged among hospital and community-acquired pathogens including Enterococcus faecium and Pseudomonas aeruginosa (Nordmann et al. 2007). P. aeruginosa infection is particularly problematic, as patients are often immune suppressed or are severely disabled and artificially ventilated. Thus, as the common antimicrobial agents begin to fail, alternative treatments which do not rely on conventional antibiotics are needed.

Another problem in treating infected chronic wounds with systemic antibiotics is that such wounds often accompany reduced local and regional circulation. Patients with venous stasis ulcers have venous thrombosis, reduced circulation and poor regional blood flow; and patients with diabetic foot ulcers suffer from poor microcirculation due to deposition of glucose and reduced circulation. Systemic antibiotics can exacerbate this problem, due to constriction of the capillaries and small blood vessels, causing a further reduction in blood flow to the wound and reduced delivery of the antimicrobial agent. Topical agents are often more effective at concentrating the antimicrobial agent at the wound site; however, they are often less effective at eliminating infection for other reasons which include reduced circulation once again. Thus, traditional therapies often leave an infected wound untreated and a patient's limb or life in danger.

In addition to poor circulation and resistant infection, many chronic wounds simply fail to heal in the face of daily wound care or treatment with advanced wound care therapy. Diabetic foot ulcers and venous stasis ulcers pose a great difficulty to patients and clinicians alike. Patients often acquire non-healing wounds due to chronic and massive atherosclerosis, venous stasis, or type II diabetes, which affects the peripheral and micro-circulation. Most often this condition results from inactivity and poor eating habits. These patients become bed ridden, immobilized, and emaciated while trying to stay off the wounds on their lower extremities, only worsening their problem of sedentary living. Clinicians frequently appeal to surgeons to bypass arteries or provide surgical coverage of wounds; however, the patients frequently have multiple co-morbidities, are not well nourished, and are poor surgical candidates. This leaves the patient and clinician with the only remaining option of treating the chronic wound with daily dressing changes, a time consuming, costly, and relatively ineffective practice. Current practice is to treat chronic wounds with daily wet-to-dry dressing changes, keeping them clean and protected until the wound heals over. However, with a lack of compliance, poor circulation, poor nutrition, non-sterile conditions, and simply the time it takes to heal wounds in this way they often stay open for years and even decades.

It has recently been shown that topical exposure of NO gas (“gNO”) to wounds such as chronic non-healing wounds can be beneficial in promoting healing and preparing the wound bed for treatment and recovery (Stenzler et al. 2006). The application of exogenous gas has been shown to reduce microbial infection, manage exudates and secretions by reducing inflammation, up regulate expression of endogenous collagenase to locally debride the wound, and regulate the formation of collagen (Stenzler et al. 2006). Furthermore, regimens have been proposed for the treatment of chronic wounds with NO g which specify high and low treatment periods to first reduce the microbial burden and inflammation and increase collagenase expression to debride necrotic tissue, and then restore the balance of NO and induce collagen expression aiding in the wound closure respectively (Stenzler et al. 2006). In fact, case studies have shown the efficacy of such a treatment by the exogenous application of gNO that was able to close a two year non-responsive, non-healing, venous stasis ulcer (Stenzler et al. 2006). The NO delivery device, however, utilized many bulky and costly components including air pump systems, gNO source cylinders, internal pressure sensors, mechanical pressure regulators, and plastic foot boot with inflatable cuff to cover the patient's lower extremity (Stenzler et al. 2006). Another drawback with the delivery of gNO is that NO rapidly oxidizes in the presence of oxygen (O₂) to form NO₂, which is highly toxic, even at low levels. A device for the delivery of NO must be anoxic, preventing NO from oxidizing to toxic NO₂ and preventing the reduction of NO which is required for the desired therapeutic effect (Stenzler et al. 2006). Thus, since NO will react with O₂ to convert to NO₂, it is desirable to have minimal contact between the gNO and the outside environment.

The antimicrobial effect of NO has been suggested by diverse observations (for example, Ghaffari et al. 2006). First, NO production by inducible NO synthases has been stimulated by proinflammatory cytokines such as IFNγ, TNF-α, IL-1, and IL-2 as well as by a number of microbial products like lipopolysaccharide (LPS) or lipoichoic acid (Fang, 1997). Infections in humans and experimental animals triggered systemic NO production as evidenced by elevated nitrates in urine and plasma. Second, elevated expression of NO in animal models improved the abilities of host to fight infectious agents and inhibited microbial proliferation, overall improving the host response (Antsey et al 1996, Evans et al 1993). Third, in-vitro studies demonstrated that inhibition of NO synthases resulted in impaired cytokine-mediated activation of phagocytic cells and reduction of bactericidal and bacteriostatic activity (Adams et al 1990). And fourth, direct administration of NO-donor compounds in-vitro, induced microbial stasis and death. Importantly, NO-dependent antimicrobial activity has been demonstrated in viruses, bacteria, fungi, and parasites (DeGroote and Fang 1995).

One of the plausible mechanisms of antimicrobial activity of NO involves the interaction of this free radical (and a reactive nitrogen intermediate) with reactive oxygen intermediates, such as hydrogen peroxide (H₂O₂) and superoxide (O₂⁻) to form a variety of antimicrobial molecular species. In addition to NO itself, these reactive antimicrobial derivatives include peroxynitrite (OONO⁻), S-nitrosothiols (RSNO), nitrogen dioxide (NO₂), dinitrogen trioxide (N₂O₃), and dinitrogen tetroxide (N₂O₄). It has been shown that these reactive intermediates target DNA, causing deamination, and oxidative damage including abasic sites, strand breaks, and other DNA alterations (Juedes et al 1996). Reactive nitrogen intermediates can also react with proteins through reactive thiols, heme groups, iron-sulfur clusters, phenolic or aromatic amino acid residues, or amines (Ischiropoulos et al 1995). Peroxinitrite and NO₂ can oxidize proteins at different sites. Additionally, NO can release iron from metalloenzymes and produce iron depletion. NO-mediated inhibition of metabolic enzymes may constitute an important mechanism of NO-induced cytostasis. Moreover, nitrosylation of free thiol groups may result in inactivation of metabolic enzymes (Fang 1997).

Several examples of the antimicrobial effects of NO have been described in the literature. Antiviral activity of NO has been described by Kawanishi (Kawanishi 1995), in in-vitro cell culture experiments, where NO donors inhibited Epstein-Barr virus late protein synthesis, amplification of DNA preventing viral replication as a result of peroxynitrite formation.

In addition, NO and superoxide produced by macrophages lead to a peroxynitrite-related anti-parasitic effect in a murine model of leishmaniasis (Augusto 1996) and the use of a topical NO donor glyceryl trinitrate was successfully used to treat cutaneous leishmaniasis (Zeina et al 1997).

Moreover, recent observations indicate that murine macrophages exert antifungal activity against candida through peroxynitrite synthesis (Vasquez-Torres et al 1996).

The antibacterial effect of NO was shown through a variety of mechanisms such as S-nitrosothiol-mediated inhibition of spore outgrowth in Bacillus cereus (Morris 1981) and several protein targets of nitrogen reactive species have been found in Salmonella typhimurium (DeGroote 1995).

Many dermatologic disorders are also amenable to topical NO therapy. Often diseases of the skin and underlying tissues are multi-factorial and can be treated topically or by elimination of an insulting agent. In many cases the mechanism of disease or its pathophysiology is associated with the complex interactions between epidermis, dermis, associated stem cells, extracellular matrix, nervous and vascular structures, complex cell signalling, and cell mediators of inflammation. In other cases the disease is directly related to an insulting agent that can be removed, eliminated, or neutralized by bioactive compounds.

Nitric oxide was formerly known as endothelial cell relaxing factor (ECRF) and acts locally to relax the cells that line blood vessels and increase the calibre of arterioles.

Further, NO is implicated in immunomodulation and T-lymphocyte responsiveness. Nitric oxide has been shown to modulate functional maturation of T lymphocytes and can enhance their activation (McInnes and Liew, 1999; Gracie et al. 1999). In mammalian cell assays, it has been shown to preferentially inhibit T-helper 1 (Th-1) clonal proliferation to antigen. The mature phenotype, in combination with specific concentrations of NO, has been shown to influence the modulatory effect of NO on human T cells. NO has also been implicated in regulation of monokine production and implicated as a factor contributing to the modulation of the immune response to different kinds of infections (McInnes and Liew, 1999).

In addition, NO has been shown to act as a proinflammatory and anti-inflammatory agent. Endogenous synthesis of NO is often correlated with production of proinflammatory cytokines. This effect can be simulated by short term topical treatment with an NO releasing agent which has been shown to have proinflammatory effects such as localized loss of Langerhans cells and apoptosis in keratinocytes in healthy skin (Cals-Grierson and Ormerod, 2004). Blockade of endogenous synthesis of NO reduces the proinflammatory effects of NO. On the other hand, NO has been shown to reduce recruitment of pro-inflammatory cells by down regulation of Endothelial Cell Adhesion Molecules such as ICAM 1 (Cals-Grierson and Ormerod, 2004). NO synthesis through Nitric oxide synthase 2 (NOS2) is partially self-regulated by the NO induced inactivation of the transcription factor NF-KB (Cals-Grierson and Ormerod, 2004).

NO can also provide protection against apoptosis through protection against oxidative stress. NO can act directly to scavenge reactive oxygen species (ROS) thereby reducing ROS mediated cell damage such as lipid peroxidation and resultant apoptosis. NO also contributes to reducing apoptosis due to oxidative stress by inducing thioredoxin expression. NO has been demonstrated to protect cells from TNF α induced apoptosis in a cGMP dependent manner (Cals-Grierson and Ormerod, 2004). There is also evidence to suggest that induction of Bcl-2 expression and suppression of caspase activation is another mechanism by which NO can protect cells from apoptosis (Cals-Grierson and Ormerod, 2004).

Dysregulation of NOS2 expression is often correlated with impairment of barrier function in dermatitis. It is postulated that this NO inhibits terminal differentiation events in keratinocytes that result in the formation of the stratum corneum (Cals-Grierson and Ormerod, 2004). NO has been shown to inhibit the transcription of some terminal differentiation proteins essential to cornification and to inactivate others. Experimental addition of exogenous NO does not amplify this effect (Cals-Grierson and Ormerod, 2004).

Oxidative damage is a time dependent process akin to rust formation on iron in the presence of oxygen. Biologically relevant free radicals are referred to as reactive oxygen species (ROS) because the most biologically significant molecules are oxygen-centered. Plants and lower organisms have evolved the biochemical machinery to make antioxidants for dealing with ROS and which prevent against their formation. Such antioxidants include vitamin E and vitamin C which are used to protect the outer layer lipophilic and hydrophilic constituents. Unfortunately, humans have lost the ability to make vitamin C, the predominant antioxidant in skin, due to a specific gene mutation. Vitamin C and other antioxidants help to protect the outer layer of cells, including biomembranes and DNA, against ROS formed endogenously by inflammatory reactions or exogenously by environmental oxidative stress (UV, ozone, etc).

Such antioxidants can be divided into enzymatic and non-enzymatic antioxidants and those which are hydrophilic and those which are lipophilic. Nitric oxide is the most naturally occurring reducing agent which is biologically available and thus can be used to prevent the action of ROS. The pathophsyiology of ROS include damage to biomembranes, DNA, enzymes and to the extracellular matrix proteins. These biological components of skin are integral to the normal form and function of skin.

In summary, several groups have developed NO producing patches or plastic containment devices holding NO g from complicated and expensive releasing devices. This, however, is a costly solution employing bulky “gas-diluting delivery systems” and “single use plastic boots”. Other devices, utilizing a chemical reaction to produce the gas, may have solved the difficulties of cost and convenience; however, are unable to provide a constant concentration over time. There remains a need for practical devices and compositions to produce NO for the treatment of wounds, microbial infections and dermatological disorders.

### SUMMARY OF THE DISCLOSURE

The present inventors have developed a composition and device in which free enzyme or bacteria combined with growth media act on substrate for the continuous production of an effective amount of nitric oxide gas (gNO). The composition is typically a time-release composition. Compositions and devices containing bacteria or enzyme isolates that act on substrate to produce gNO are effective in the treatment of wounds, microbial infections and/or dermatological disorders.

The inventors have designed a device that uses microorganisms for sustained production of controlled amounts of nitric oxide (NO). Biosynthesis of NO through the denitrification pathway from nitrate is a well known mechanism in microorganisms and this application provides the first disclosure of methods of medical treatment of wounds, microbial infections and/or dermatological disorders using such gas. Some lactobacilli reduce nitrate (NO₃⁻) to nitrite (NO₂⁻) and NO under anaerobic conditions (nitrate reductase) (Wolf et al. 1990). Other microorganisms produce NO by metabolism of L-arginine (NOS enzyme) nitrate in the growth medium under anaerobic conditions (Xu & Verstraete 2001).

Immobilized bacteria or free enzyme, in the presence of precursor substrates, can produce NO over the desired therapeutic time and at therapeutically relevant levels. The therapeutic capability of the bacteria or enzyme is maintained over the period of time in which they have sufficient nutrients, are not surrounded by excess waste, and have the substrate and cofactors required to be biochemically efficient at producing the therapeutic gas.

Accordingly, the present application discloses methods, compositions and devices for treating wounds, microbial infections and/or dermatological disorders using a topical source of nitric oxide.

In an aspect, the application provides a composition for delivering nitric oxide gas topically to affected tissue. In an embodiment, the application provides a composition for delivering nitric oxide gas to affected tissue comprising (a) an isolated enzyme or a live cell expressing an endogenous enzyme, the enzyme (i) having activity that converts a nitric oxide gas precursor to nitric oxide gas or (ii) having activity on a substrate that produces a catalyst that causes the conversion of the nitric oxide gas precursor to nitric oxide gas, or (b) a live cell producing a catalyst for converting a nitric oxide gas precursor to nitric oxide gas; and a carrier. In an embodiment, the nitric oxide gas precursor is present on the tissue of the subject, for example, in the form of nitrate produced from sweat. In another embodiment, the composition further comprises a nitric oxide gas precursor. In yet another embodiment, the carrier comprises a matrix.

In another aspect, the application provides a device for delivering nitric oxide gas topically to affected tissue. In an embodiment, the application provides a device for delivering nitric oxide gas to affected tissue comprising a casing having a barrier surface and a contact surface that is permeable to nitric oxide gas; and a composition in the casing that is comprised of i) a nitric oxide gas precursor, and ii) (a) an isolated enzyme or a live cell expressing an endogenous enzyme, the enzyme 1) having activity that converts the nitric oxide gas precursor to nitric oxide gas or 2) having activity on a substrate that produces a catalyst that causes the conversion of the nitric oxide gas precursor to nitric oxide gas, or (b) a live cell producing a catalyst for converting the nitric oxide gas precursor to nitric oxide gas.

In an embodiment, the affected tissue comprises a wound, a microbially-infected tissue and/or tissue from a subject having a dermatological disorder. In one embodiment, the affected tissue is skin and the casing is suitable for topical administration to the skin.

In another embodiment, the device further comprises a nitric oxide gas concentrating agent.

In yet another embodiment, the casing comprises a plurality of layers. In one embodiment, the layers include a barrier layer; a contact layer; and an active layer. In another embodiment, the active layer comprises the composition; the barrier layer comprises the barrier surface and the contact layer comprises the contact surface. In a further embodiment, the casing also includes a reservoir layer. In one embodiment, the reservoir layer comprises the nitric oxide gas precursor. In yet another embodiment, the casing also includes a trap layer. In one embodiment, the trap layer comprises the nitric oxide gas concentrating agent.

In another aspect, the application provides methods and uses of a device or composition of the application for treatment of a wound, a microbial infection and/or a dermatological disorder in a subject in need thereof.

In one aspect, the application provides a method for treatment of a wound, a microbial infection and/or a dermatological disorder in a subject in need thereof comprising

contacting affected tissue with a casing permeable to nitric oxide gas, the casing containing a plurality of inactive agents that, upon activation, react to produce nitric oxide gas;

activating the inactive agents to produce nitric oxide gas,

wherein the nitric oxide gas communicates through the casing and contacts the affected tissue to treat the wound, microbial infection and/or dermatological disorder in the subject in need thereof.

In another aspect, the application provides a method for treating a wound, a microbial infection and/or a dermatological disorder in a subject in need thereof comprising

contacting affected tissue with a nitric oxide gas releasing composition, the composition containing a plurality of inactive agents that, upon activation, react to produce nitric oxide gas;

activating the inactive agents to produce nitric oxide gas,

wherein the nitric oxide gas contacts the affected tissue for treating the wound, the microbial infection or dermatological disorder in the subject in need thereof.

In an embodiment, the inactive agents are separated and activation of the inactive agents comprises combining the separated agents together by mixing the separated agents only after an applied pressure or temperature. In another embodiment, the inactive agents are dehydrated agents and activation of the inactive agents comprises hydration.

In another embodiment, the inactive agents comprise i) a nitric oxide gas precursor, ii) (a) an isolated enzyme or a live cell expressing an endogenous enzyme, the enzyme having activity that converts the nitric oxide gas precursor to nitric oxide gas or having activity on a substrate that produces a catalyst that causes the conversion of the nitric oxide gas precursor to nitric oxide gas or (b) a live cell producing a catalyst for converting the nitric oxide gas precursor to nitric oxide gas.

In yet another aspect, the disclosure provides a method for treatment of a wound, a microbial infection and/or a dermatological disorder in a subject in need thereof comprising exposing affected tissue to a device or composition of the application, wherein NO produced by the device or composition contacts the affected tissue for a treatment period without inducing toxicity to the subject or healthy tissue. The treatment period will depend on the type of device or composition used. For example, for a device described herein, the treatment period typically is from about 1 to 24 hours, preferably about 6-10 hours and more preferably about 8 hours. For a composition contained in a patch, the treatment period typically is from about 1 to 8 hours. For a cream composition, the cream is typically applied one to three times daily. For a mask composition, the treatment period is typically from about 1 to 8 hours, optionally, 1-2 hours.

In yet a further embodiment, the NO is produced by the device or composition in an amount suitable for the particular use and can range from 1 to 1000 parts per million volume (ppmv). In one embodiment, the NO produced by the device or composition for wounds is from about 1 to 1000 ppmv. In another embodiment, the NO produced by the device or composition for infections is from about 150 to 1000 ppmv. In yet another embodiment, the NO produced by the device or composition for dermatological disorders is from about 5 to 500 ppmv.

In another aspect, there is provided a method for treatment of a wound in a subject in need thereof comprising:

first exposing the wound to a device of the application to produce a high concentration of nitric oxide gas that contacts the wound for a first treatment period without inducing toxicity to the subject or healthy tissue; and

second exposing the wound to a second device of the application to produce a low concentration of nitric oxide gas that contacts the wound for a second treatment period.

In a further aspect, the disclosure provides a method of improving red meat product shelf life, preservation, or physical appearance comprising exposing the red meat product to a device of the application, wherein NO contacts the red meat product.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the disclosure will now be described in relation to the drawings in which:

FIG. 1 shows the concentration of Nitric Oxide gas (gNO) released by MRS agar growing Lactobacillus fermentum (ATCC 11976) supplemented with several concentrations of NaNO₂. The concentration of gNO produced by MRS medium growing Lactobacillus fermentum (ATCC 11976) supplemented with a 40 cm² Nitro-Dur 0.8 mg/hr nitro-glycerine transdermal patch (GTN) (Key Pharmaceuticals) is also shown. Measurements were made after 20 hours of growth at 37° C. without shaking.

FIG. 2 shows nitric oxide gas (gNO) released by medium growing Lactobacillus fermentum (ATCC 11976) with the indicated concentrations of NaNO₂ or Escherichia coli BL21 (pnNOS) (pGroESL) with the indicated cofactors. Measurements were made after 20 hours of growth at 37° C. without shaking.

FIG. 3A shows nitric oxide gas released by the medium growing either Lactobacillus plantarum LP80, Lactobacillus fermentum (ATCC 11976), Lactobacillus fermentum (NCIMB 2797) or Lactobacillus fermentum (LMG 18251) with the indicated concentrations of KNO₃ or NaNO₂. Measurements were made after 20 hours of growth at 37° C. without shaking. FIG. 3B shows nitrite released by the medium growing either Lactobacillus plantarum LP80, Lactobacillus fermentum (ATCC 11976), Lactobacillus fermentum (NCIMB 2797) or Lactobacillus fermentum (LMG 18251) with the indicated concentrations of KNO₃ or NaNO₂. Measurements were made after 20 hours of growth at 37° C. without shaking. FIG. 3C shows nitrate released by the medium growing either Lactobacillus plantarum LP80, Lactobacillus fermentum (ATCC 11976), Lactobacillus fermentum (NCIMB 2797) or Lactobacillus fermentum (LMG 18251) with the indicated concentrations of KNO₃ or NaNO₂. Measurements were made after 20 hours of growth at 37° C. without shaking.

FIG. 4A is a graph that shows the pH of the medium growing Lactobacillus fermentum (ATCC 11976) with the indicated concentrations of NaNO₂ and 20 g/L (no glucose added) or 100 g/L (glucose added) glucose. Measurements were made after the indicated number of hours at 37° C. without shaking. FIG. 4B is a graph that shows the optical density of the medium growing Lactobacillus fermentum (ATCC 11976) with the indicated concentrations of NaNO₂ and 20 g/L (no glucose added) or 100 g/L (glucose added) glucose. Measurements were made after 3, 4, 5, 6, and 20 hours at 37° C. without shaking. FIG. 4C is a nitric oxide gas released by the medium growing Lactobacillus fermentum (ATCC 11976) with the indicated concentrations of NaNO₂ and 20 g/L (no glucose added) or 100 g/L (glucose added) glucose. Measurements were made after the indicated number of hours at 37° C. without shaking.

FIG. 5 shows a graphical representation of the relative quantity of nitric oxide gas (NO g), as represented by area under the curve, produced by strains of Lactobacillus fermentum grown in MRS media at 37° C. for 20 hours.

FIG. 6 shows a repeat of the relative quantity of nitric oxide gas (NO g), as represented by area under the curve, produced by strains of Lactobacillus fermentum grown in MRS media at 37° C. for 20 hours.

FIG. 7 shows the head gas pressure (kPa) in the vessel where strains of Lactobacillus fermentum were grown in MRS media at 37° C. for 20 hours.

FIG. 8 shows nitrate (NO₃) produced by strains of Lactobacillus fermentum grown in MRS media at 37° C. for 20 hours.

FIG. 9 shows nitrite (NO₂) produced by strains of Lactobacillus fermentum grown in MRS media at 37° C. for 20 hours.

FIG. 10 shows nitric oxide gas produced by Lactobacillus reuteri

(NCIMB 701359), Lactobacillus reuteri (LabMet) and Lactobacillus fermentum (ATCC 11976) in the presence of ½ patch of nitroglycerin (first 4 columns) or in the presence of ½ patch of nitroglycerin with the addition of P450 or gluthathione-5-transferase inhibitors (last 3 columns).

FIG. 11 shows a multilayered nitric oxide producing medical device.

FIG. 12 shows a simple single layered medical device.

FIG. 13 shows another simple layered medical device.

FIG. 14 shows yet another simple layered medical device.

FIG. 15 shows the bactericidal effect of gNO-producing patches on E. Coli. Whereas bacterial count remained stable after an 8-hour treatment with controls (squares), in the presence of gNO no colonies were detected after 6 hours (diamonds) (upper panel). Levels of gNO produced by active patches (diamonds) or controls (squares) were monitored hourly (lower panel).

FIG. 16 shows the bactericidal effect of gNO-producing patches on S. Aureus. Whereas bacterial count remained stable after an 8-hour treatment with controls (squares), in the presence of gNO no colonies were detected after 6 hours (diamonds) (upper panel). Levels of gNO produced by active patches (diamonds) or controls (squares) were monitored hourly (lower panel).

FIG. 17 shows the bactericidal effect of gNO-producing patches on P. Aeruginosa. Whereas bacterial count remained stable after an 8-hour treatment with controls (squares), in the presence of gNO no colonies were detected after 6 hours (diamonds) (upper panel). Levels of gNO produced by active patches (diamonds) or controls (squares) were monitored hourly (lower panel).

FIG. 18 shows the bactericidal effect of gNO-producing patches on Acinetobacter baumannii. Whereas bacterial count remained stable after a 6-hour treatment with controls (squares), in the presence of gNO less than 10 colonies were detected after the same period (diamonds) (upper panel). Levels of gNO produced by active patches (diamonds) or controls (squares) were monitored hourly (lower panel).

FIG. 19 shows the fungicidal effect of gNO-producing patches on Trichophyton rubrum. Whereas fungal growth remained constant after an 8-hour treatment with controls (gray), no colonies were detected after 8 hours (black) in the presence of gNO. Levels of gNO produced by active patches (black) or controls (grey) were monitored hourly.

FIG. 20 shows the fungicidal effect of gNO-producing patches on Trichophyton mentagrophytes. Whereas fungal growth remained constant after an 8-hour treatment with controls (gray), no colonies were detected after 6 hours (black) in the presence of gNO. Levels of gNO produced by active patches (black) or controls (grey) were monitored hourly for 7 hours.

FIG. 21 shows the bactericidal effect of gNO-producing patches on Methicillin-resistant Staphylococcus aureus (MRSA). Whereas bacterial growth remained constant after a 6-hour treatment with controls (gray), no colonies were detected after 6 hours (black) in the presence of gNO. Levels of gNO produced by active dressing (black) or controls (grey) were monitored hourly for 6 hours.

FIG. 22 (left) shows the bacteriostatic effect of gNO-producing patches on E. Coli. Treatment of E. Coli plates with gNO-producing patches inhibited the growth of colonies as compared to control patches. FIG. 22 (middle) shows the bacteriostatic effect of gNO-producing patches on S. Aureus. Treatment of S. Aureus plates with gNO-producing patches reduced the growth of colonies as compared to control patches. FIG. 22 (right) shows the bacteriostatic effect of gNO-producing patches on P. Aeruginosa. Treatment of P. Aeruginosa plates with gNO-producing patches reduced the growth of colonies as compared to control patches.

FIG. 23 shows the effect of gNO-treatment as compared to vehicle control in the 4 experimental conditions as seen daily by morphometric analysis of the wounds. Wound healing was monitored daily and photographic records were kept for morphometric analysis. The diameters of each wound and the 6 mm-diameter references (green or red stickers) were determined using computer software by the longest measurement to correct for plane inclinations. The wound areas were calculated by multiplying the area corresponding to a 6 mm-diameter circle by the ratio of the squares of the wound diameter-to-reference diameter.

FIG. 24 shows the appearance of infected wounds at days 1, 13, and 20 post-surgery. Ischemic wounds are indicated by “I” while non-ischemic wounds are indicated by an “N”. Wound healing was monitored daily and photographic records were kept for morphometric analysis. Starting on the day of surgery, photographs were taken of the wounds on each ear. A group picture with all 4 wounds was taken first, followed by pictures of each wound.

FIG. 25 shows a Cox proportional hazard regression comparing treated vs untreated wounds. The data was graphed using EpiInfo software from the CDC. It represents time to event (wound closure) for all the wounds generated and treated in the pilot study. Dark line is gNO treated wounds (16 wounds), Gray line is untreated (16 wounds). See also Table 7.

FIG. 26 shows a Kaplan-meier plot of wound healing data from pilot study. The data was graphed using EpiInfo software from the CDC. It represents time to event (wound closure) for all the wounds generated and treated in the pilot study. Dark line is gNO treated wounds (16 wounds), Gray line is untreated (16 wounds). See also Table 8.

FIG. 27 shows the generation of gNO measured hourly in the presence of porcine liver esterase, sodium nitrite, and various ester substrates. A minimum target production was achieved one hour after path activation. No gNO was detected using controls in which neither substrate (triacetin) nor enzyme were present. The best substrates for porcine liver esterase are triacetin and ethyl acetate.

FIG. 28 shows the generation of gNO measured hourly in the presence of candida rugosa lipase (“CRL”), sodium nitrite, and various ester substrates. A minimum target gNO production of 200 ppmV was achieved with triacetin as a substrate, one hour after the reaction was started.

FIG. 29 shows the generation of gNO measured hourly in the presence of triacetin, sodium nitrite, and various enzymes. No gNO production was obtained in the absence of substrate or enzyme. Candida rugosa lipase and porcine liver esterase are the best enzymes for triacetin.

FIG. 30 shows the generation of gNO analyzed in the presence of sodium nitrite, porcine liver esterase and varying concentrations of triacetin. No gNO production was observed in the absence of enzyme or substrate (triacetin).

FIG. 31 shows the generation of gNO evaluated hourly in 4 patches containing triacetin, CRL, alginate microbeads and sodium nitrate. A target production gNO of over 200 ppmV was reached 2 hours after patch activation and it was sustained up to 30 hours.

### DETAILED DESCRIPTION OF THE DISCLOSURE

The present application provides a topical device and a topical composition capable of continually producing nitric oxide production and its methods and uses for administration of nitric oxide to treat a wound, microbial infection and/or dermatological disorder.

### Compositions and Devices

In one aspect, the disclosure provides a topical composition comprising (a) an isolated enzyme or a live cell expressing an endogenous enzyme, the enzyme having activity that converts the nitric oxide gas precursor to nitric oxide gas or having activity on a substrate that produces a catalyst that causes the conversion of the nitric oxide gas precursor to nitric oxide gas, or (b) a live cell producing a catalyst for converting the nitric oxide gas precursor to nitric oxide gas. In one embodiment, the nitric oxide gas precursor is present on the tissue, for example, from nitrate produced in sweat. In another embodiment, the composition further comprises a nitric oxide gas precursor.

The term “topical composition” as used herein refers to any substance that comprises the enzyme, live cell or catalyst and optionally, the nitric oxide precursor, and can be applied directly or locally to affected tissue and acts locally on the affected tissue. Optionally, the affected tissue is skin. In one embodiment, the topical composition is a cream, slab, gel, hydrogel, dissolvable film, spray, paste, emulsion, patch, liposome, balm, powder or mask or a combination thereof. In another embodiment the composition is two separate parts.

In one embodiment, the composition further comprises a matrix. A person skilled in the art can readily determine a suitable matrix for topical application. The matrix optionally includes, without limitation, a natural polymer, such as alginate, chitosan, gelatin, cellulose, agarose, locust bean gum, pectin, starch, gellan, xanthan and agaropectin; a synthetic polymer, such as polyethyleneglycol (PEG), polyacrylamide, polylacticacid (PLA), thermoactivated polymers and bioadhesive polymers; a gel or hydrogel, such as petroleum jelly, intrasite, and lanolin or water-based gels; hydroxyethylcellulose and ethyleneglycol dglycidylether (EDGE); a dissolvable film polymer such as hydroxymethylcellulose; a microcapsule or liposome; and lipid-based matrices. Intrasite is a colourless transparent aqueous gel, which typically contains a modified carboxymethylcellulose (CMC) polymer together with propylene glycol as a humectant and preservative, optionally 2.3% of a modified carboxymethylcellulose (CMC) polymer together with propylene glycol (20%). When placed in contact with affected tissue, a dressing absorbs excess exudate and produces a moist environment at the surface of the tissue, without causing tissue maceration.

Other matrix components, include, without limitation, vitamin A, vitamin B, vitamin C, vitamin D, vitamin E, vitamin K, zinc oxide, ferulic acid, caffeic acid, glycolic acid, lactic acid, tartaric acid, salicylic acid, stearic acid, sodium bicarbonate, salt, sea salt, aloe vera, hyaluronic acid, glycerine, silica silylate, polysorbate, purified water, witch hazel, coenzyme, soy protein (hydrolysed), hydrolyzed wheat protein, methyl & propyl paraben, allantoin, hydrocarbons, petroleum jelly, rose flower oil (rosa damascens), lavender and other typical moisturizers, softeners, antioxidants, anti-inflammatory agents, vitamins, revitalizing agents, humectants, coloring agents and/or perfumes known in the art.

In an embodiment, the composition is applied to a bandage, dressing or clothing.

In another aspect, the application provides a device comprising the compositions described herein. In one embodiment, the device comprises a casing comprising a barrier surface and a contact surface, said contact surface being permeable to nitric oxide gas, wherein the casing comprises a composition described herein, and the composition is located between the barrier surface and the contact surface. The barrier surface is optionally connected to the contact surface so that the barrier surface and contact surface define a cavity in which the composition is located. Typically the barrier surface is connected to the contact surface proximate to the perimeter of the contact surface so that the barrier surface surrounds the perimeter thereof, thereby requiring NO gas to leave only through the contact surface. In an embodiment, the application provides a device for delivering nitric oxide gas to affected tissue, comprising
a casing comprising a barrier surface and a contact surface, said contact surface being permeable to nitric oxide gas and
a composition in the casing, the composition comprising i) a nitric oxide gas precursor, and ii) (a) an isolated enzyme or a live cell expressing an endogenous enzyme, the enzyme having activity that converts the nitric oxide gas precursor to nitric oxide gas or having activity on a substrate that produces a catalyst that causes the conversion of the nitric oxide gas precursor to nitric oxide gas, or (b) a live cell producing a catalyst for converting the nitric oxide gas precursor to nitric oxide gas.

In one embodiment, the casing separates the composition from the tissue and the casing is impermeable to the composition.

The term “affected tissue” as used herein refers to any tissue, optionally skin, having a wound, a microbial infection and/or a dermatological disorder. For example, affected tissue includes abnormal tissue or damaged tissue, i.e. tissue that is pathologically, histologically, morphologically or molecularly different than normal tissue and that would benefit from NO treatment.

The term “casing” as used herein means a shell that retains the composition, and wholly or partially covers the composition. In one embodiment, the casing is a series or plurality of layer(s), for example, flexible and/or rigid laminate. In another embodiment, the casing is a bag or a container. The term “in the casing” as used herein means wholly or partially covering and retaining the composition such that the composition is separated from tissue.

The term “contact surface” as used herein means the surface of the casing that directly interacts with the tissue and can be made of any suitable material such as a non-occlusive dressing.

The term “barrier surface” as used herein means the surface of the casing that is not directly contacting the tissue, that is, the entire surface of the casing except for the contact surface which directly contacts the tissue. The barrier surface may be permeable or impermeable to oxygen. The barrier surface may be made of any suitable material such as plastic. In another embodiment, the barrier surface comprises an adhesive layer that adheres to the tissue surrounding the affected tissue. In a particular embodiment, the barrier surface is oxygen permeable, protects the tissue or skin and adheres to the tissue or skin.

In another embodiment, the layers of the casing comprise a barrier layer, a contact layer and an active layer. In a particular embodiment, the active layer comprises the composition, the barrier layer comprises the barrier surface and the contact layer comprises the contact surface. In another embodiment, the casing further comprises a reservoir layer. In one embodiment, the active layer comprises the cell or enzyme and the reservoir layer comprises the nitric oxide gas precursor.

In a further embodiment, the casing further comprises a trap layer. In one embodiment the trap layer comprises the nitric oxide gas or radical concentrating substance.

The term “nitric oxide gas” or “gNO” or “NO g” as used herein refers to the chemical compound NO and is also commonly referred to as nitric oxide radical.

The term “enzyme” as used herein is intended to include any enzyme or fragment thereof capable of converting a nitric oxide precursor to nitric oxide gas either directly or through the production of a catalyst that causes the conversion of the nitric oxide gas precursor to nitric oxide gas.

In one embodiment, the enzyme is a glutathione S-transferase (GST) or cytochrome P450 system (P450).

In another embodiment, the enzyme is nitric oxide synthase enzyme (NOS) or nitric oxide reductase (NiR). In an embodiment, the enzyme is all or part of the nitric oxide synthase enzyme having NOS activity. In a particular embodiment, the NOS comprises the amino acid sequence as shown in SEQ ID NO:1 or Table 1. In another embodiment, the enzyme is all or part of the nitric oxide reductase having NIR activity. In a particular embodiment the NiR comprises several subunits with amino acid sequences as shown in SEQ ID NOs:2-5 or Table 1. The enzyme optionally is contained in a protein fraction isolated from cells.

The term “catalyst” or “nitric oxide gas precursor reducing agent” as used herein means a substance that causes the conversion of the nitric oxide gas precursor to nitric oxide gas optionally through a dismutation reaction. Further, the catalyst is readily produced through the reaction of an enzyme with a substrate. In another embodiment, the catalyst is lactic acid, acetic acid, sulfuric acid, hydrochloric acid or other weaker organic acids. In a particular embodiment, the catalyst is lactic acid. In another embodiment, the catalyst comprises protons. In one embodiment, the protons are a product of the reaction of the enzyme with the substrate. The term “product of the reaction” as used herein includes both products and/or by-products of the enzyme reaction.

In one embodiment, the catalyst producing enzyme is from a bromelain solution, an extract optionally from pineapple or is a genetically engineered bromelain protease enzyme. Bromelain as used herein refers to a crude, aqueous extract from the stems and immature fruits of pineapples (Ananas comosus Merr., mainly var. Cayenne from the family of bromeliaceae), constituting an unusually complex mixture of different thiol-endopeptidases and other not yet completely characterized components such as phosphatases, glucosidases, peroxidases, cellulases, glycoproteins and carbohydrates, among others. In addition, bromelain contains several proteinases inhibitors. In one embodiment, the enzyme and substrate that produce a catalyst comprises bromelain, which contains both enzyme and substrate, bromelain and protein, such as gelatin.

In another embodiment, the enzyme and substrate that produce a catalyst comprise lipase and lipid (for example, a triglyceride), protease and protein, trypsin and protein, chymotrypsin and protein, esterase and ester, lipase and ester, or esterase and triglyceride. In one embodiment, the enzyme is a lipase or esterase, optionally candida rugossa lipase, porcine liver esterase, Rhisopus oryzae esterase or Porcine pancrease lipase. In another embodiment, the substrate is a triglyceride or ester, optionally triacetin, tripropyrin, tributyrin, ethyl acetate, octyl acetate, butyl acetate or isobutyl acetate. In another embodiment, the enzyme and substrate that produce a catalyst comprise lactose dehydrogenase and lactose, papain and protein, pepsin and protein or pancreatin and soy protein.

The term “nitric oxide gas precursor” as used herein means any substrate that may be converted into nitric oxide gas. Accordingly, in an embodiment, the nitric oxide gas precursor is a substrate for enzymatic production of nitric oxide. In one embodiment, the nitric oxide gas precursor is L-arginine. In another embodiment, the nitric oxide gas precursor is nitrate or a salt thereof, such as potassium nitrate, sodium nitrate or ammonium nitrate or other nitrate. In one embodiment, the nitrate is nitrate produced from sweat. In yet another embodiment, the nitric oxide gas precursor is a nitrite or salt thereof, such as potassium nitrite or sodium nitrite. In one embodiment, 1-50 mmol of sodium nitrite are used. In another embodiment, 30 mmol of sodium nitrite are used. In yet another embodiment, the nitric oxide gas precursor is a nitric oxide donor, optionally nitroglycerine or isosorbide nitrate. In one embodiment, the enzyme comprises NiR and the nitric oxide gas precursor comprises potassium nitrite or the enzyme comprises NOS and the nitric oxide precursor comprises L-arginine. In another embodiment, the enzyme comprises a nitrate reductase and the nitric oxide gas precursor is a nitrate salt. In yet another embodiment, the nitric oxide gas precursor is a nitro-glycerine or nitrate located in an eluting transdermal system, such as a patch. In a further embodiment, the enzyme is glutathione S-transferase (GST) or cytochrome P450 system (P450) and the nitric oxide gas precursor is nitroglycerine, a nitrosorbide dinitrate, or a nitrate.

Enzyme or catalyst activity is readily determined by an assay measuring the nitric oxide gas product. The preferred NO assay is a chemiluminescent assay. A sample containing nitric oxide is mixed with a large quantity of ozone. The nitric oxide reacts with the ozone to produce oxygen and nitrogen dioxide. This reaction also produces light (chemiluminescence), which can be measured with a photodetector. The amount of light produced is proportional to the amount of nitric oxide in the sample.

The disclosure also includes modified NOS and NIR polypeptides which have sequence identity of at least about: >20%, >25%, >28%, >30%, >35%, >40%, >50%, >60%, >70%, >80% or >90% more preferably at least about >95%, >99% or >99.5%, to SEQ ID NO:1 and SEQ ID NOs:2-5 respectively. Modified polypeptide molecules are discussed below.

Identity is calculated according to methods known in the art. Sequence identity is most preferably assessed by the BLAST version 2.1 program advanced search (parameters as above). BLAST is a series of programs that are available online from the National Center for Biotechnology Information (NCBI) of the U.S. National Institutes of Health. The advanced BLAST search is set to default parameters. (i.e. Matrix BLOSUM62; Gap existence cost 11; Per residue gap cost 1; Lambda ratio 0.85 default).

References to BLAST searches are: Altschul, S. F., Gish, W., Miller, W., Myers, E. W. & Lipman, D. J. (1990) “Basic local alignment search tool.” J. Mol. Biol. 215:403-410; Gish, W. & States, D. J. (1993) “Identification of protein coding regions by database similarity search.” Nature Genet. 3:266-272; Madden, T. L., Tatusov, R. L. & Zhang, J. (1996) “Applications of network BLAST server” Meth. Enzymol. 266:131-141; Altschul, S. F., Madden, T. L., Schäffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402; Zhang, J. & Madden, T. L. (1997) “PowerBLAST: A new network BLAST application for interactive or automated sequence analysis and annotation.” Genome Res. 7:649-656.

Preferably about: 1, 2, 3, 4, 5, 6 to 10, 10 to 25, 26 to 50 or 51 to 100, or 101 to 250 nucleotides or amino acids are modified. The disclosure includes polypeptides with mutations that cause an amino acid change in a portion of the polypeptide not involved in providing activity or an amino acid change in a portion of the polypeptide involved in providing activity so that the mutation increases or decreases the activity of the polypeptide.

In one embodiment, the enzyme has animal, plant, fungal or bacterial origin.

In another embodiment, the composition further comprises an enzyme cofactor. Enzyme cofactors useful in the device include tetrahydrobiopterin (H₄B), calcium ions (Ca²⁺), flavin adenine dinucleotide (FAD), flavin mononucleotide (FMN), beta-nicotinamide adenine dinucleotide phosphate reduced (NADPH), molecular oxygen O₂ and calmodulin.

The compositions and devices described herein can be made more effective by the addition of bioactive molecules that react with reactive oxygen species (ROS) which normally consume nitric oxide. Bioactive low molecular weight (LMWT) and enzymatic antioxidants can prevent the consumption of NO by ROS (Serarslan et al. 2007). The reaction between NO and ROS forms peroxynitrite (ONO₂⁻), disabling NO and preventing its normal physiologic action. The use of antioxidants, either added pure or produced in an in-situ reaction between cell or enzyme isolates and substrate, can prevent the consumption of NO by ROS providing an improved NO delivery formulation for topical application.

Accordingly, in another embodiment, the composition further comprises an antioxidant for maintaining a reducing environment. The antioxidant may be expressed by the live cell or produced in a reaction between a second enzyme, either added or expressed by the live cell, and an antioxidant precursor. In one embodiment, the antioxidant is caffeic acid, ferulic acid, or chlorogenic acid. In another embodiment, the antioxidant is dithionite, methaquinone or ubiquinone. In yet another embodiment, the antioxidant is a vitamin, optionally, vitamin K, vitamin E or vitamin C.

The term “live cell” as used herein means any type of cell that is capable of converting nitric oxide precursor to nitric oxide at the site of action. In one embodiment, the cell is a human, bacterial or yeast cell. In another embodiment the cell is a probiotic microorganism of the genus Lactobacillus, Bifidobacteria, Pediococcus, Streptococcus, Enterococcus, or Leuconostoc. In one embodiment, the cell is Lactobacillus plantarum, Lactobacillus fermentum, Pediococccus acidilactici, or Leuconostoc mesenteroides. In another embodiment, the cell is a yeast cell selected from the group consisting of one or more of a Torula species, baker's yeast, brewer's yeast, a Saccharomyces species, optionally S. cerevisiae, a Schizosaccharomyces species, a Pichia species optionally Pichia pastoris, a Candida species, a Hansenula species, optionally Hansenula polymorpha, and a Klyuveromyces species, optionally Klyuveromyces lactis. In one embodiment, the cell is a bacteria that produces a mild acid, including without limitation, lactic acid, acetic acid, malic acid and tartaric acid. In yet another embodiment, the cell is a lactic acid bacteria (LAB) or an acetobacter, such as acetobacter pastureianis.

In a further embodiment, the cell is a genetically engineered cell expressing an enzyme that is capable of converting a nitric oxide gas precursor to nitric oxide gas. In one embodiment, the cell is a genetically engineered yeast expressing NOS or NiR enzyme. In another embodiment, the cell is a genetically engineered bacteria expressing NOS or NiR enzyme. In yet another embodiment, the cell is Escherichia coli BL21 (nNOSpCW), an E. coli or Lactobacillus strain expressing bacterial nitrite reductases, optionally a copper-dependant nitrite reductase from Alcaligenes faecalis S-6 or an E. coli or Lactobacillus strain expressing a cytochrome cd1 nitrite reductase from Pseudomonas aeruginosa.

A person skilled in the art would be able to quantify the amount of NO produced by a cell or enzyme. For example, Kikuchi et al. describe a method for the quantification of NO using horseradish peroxidise in solution (Kikuchi et al. 1996). Archer et al reviewed the measurement of NO in biological systems and found that the chemiluminescence assay is the most sensitive technique with a detection threshold of roughly 20 pmol (Archer 1993; Michelakis & Archer 1998).

In another embodiment, the cell is microencapsulated. In one embodiment, the microcapsule comprises Alginate/Poly-l-lysine/Alginate (APA), Alginate/Chitosan/Alginate (ACA), or Alginate/Genipin/Alginate (AGA) membranes. In another embodiment, the microcapsule comprises Alginate/Poly-l-lysine/Pectin/Poly-l-lysine/Alginate (APPPA), Alginate/Poly-l-lysine/Pectin/Poly-l-lysine/Pectin (APPPP), Alginate/Poly-L-lysine/Chitosan/Poly-l-lysine/Alginate (APCPA), alginate-polymethylene-co-guanidine-alginate (A-PMCG-A), hydroxymethylacrylate-methyl methacrylate (HEMA-MMA), Multilayered HEMA-MMA-MAA, polyacrylonitrilevinylchloride (PAN-PVC), acrylonitirle/sodium methallylsuflonate (AN-69), polyethylene glycol/poly pentamethylcyclopentasiloxane/polydimethylsiloxane (PEG/PD5/PDMS) or poly N,N-dimethyl acrylamide (PDMAAm) membranes. In a further embodiment, the microcapsule comprises alginate, hollow fiber, cellulose nitrate, polyamide, lipid-complexed polymer, a lipid vesicle a siliceous encapsulate, cellulose sulphate/sodium alginate/polymethylene-co-guanidine (CS/A/PMCG), cellulose acetate phthalate, calcium alginate, k-carrageenan-Locust bean gum gel beads, gellan-xanthan beads, poly(lactide-co-glycolides), carageenan, starch polyanhydrides, starch polymethacrylates, polyamino acids or enteric coating polymers.

In another embodiment, the cell or enzyme of the composition is immobilized in a reservoir, such as a slab. In one embodiment, the reservoir or slab comprises a polymer. In a particular embodiment, the polymer is a natural polymer such as alginate, chitosan, agarose, agaropectin, or cellulose.

In yet another embodiment, the composition further comprises growth media for cells. Typical growth media include MRS broth, LB broth, glucose, or carbon source containing growth media. The choice of growth media depends on the requirements of the particular cells of the composition of the device of the application.

In a further embodiment, a reducing agent is added. In one embodiment, the reducing agent leads to improved stoichiometry and additional NO production. In an embodiment, the reducing agent is sodium iodide (NaI).

In a further embodiment, the device further comprises a nitric oxide gas or radical concentrating agent. The term “nitric oxide gas or radical concentrating agent” as used herein is intended to cover any substance that is capable of collecting and concentrating the nitric oxide gas for application to the affected tissue.

In one embodiment, the nitric oxide gas or radical concentrating agent comprises lipid or lipid-like molecules. The term “lipids and lipid-like molecules” as used herein mean substances that are fat soluble. An example of a lipid-like molecule is a lipopolysaccharide which is a lipid and a carbohydrate molecule joined by a covalent bond.

In another embodiment, the nitric oxide gas or radical concentrating agent comprises hydrocarbon or hydrocarbon-like molecules. The term “hydrocarbon” as used herein means a hydrogen and carbon containing compound which has a carbon “backbone” and bonded hydrogens, sulfur or nitrogen (impurities), or functional groups. The term “hydrocarbon-like molecule” refers to a molecule that has a carbon backbone and contains hydrogens but may have a complex and highly bonded or substituted structure. Both hydrocarbons and hydrocarbon-like molecules are lipid soluble.

In yet another embodiment, the nitric oxide gas or radical concentrating agent comprises a spacer, a gas cell containing structure or a sponge.

In one aspect, the nitric oxide gas precursor and the composition comprising live cells, enzyme or catalyst are separated until use. Accordingly in one embodiment of the composition of the application, the nitric oxide gas precursor and composition comprising live cell, enzyme or catalyst are kept separate and are mixed immediately prior to use. In an embodiment of the device, the active layer and reservoir layer are separated by a separator. The separator is a physical barrier, optionally made from plastic or other suitable material, typically between the active layer and reservoir layer, that prevents the contents of the active layer and reservoir layer from combining. In another embodiment, the casing further comprises at least one valve connecting the active layer and the reservoir layer, wherein the valve has an initial closed position in which the cell or enzyme are separate from the precursor and an open position in which the active layer and reservoir layer are in fluid communication, and the cell or enzyme precursor are permitted to flow between the layers. In another embodiment, the valve comprises a one-way valve, and wherein in the open position either the enzyme or cell or the precursor is permitted to flow between the layers. In another embodiment, the valve comprises a pressure actuated valve that is actuable from the closed position to the open position by compression of the device, optionally manual compression. In yet a further embodiment, the composition alone or in the device is dehydrated and is inactive until hydration.

### Methods and Uses

In another aspect, the application provides the use of a device or composition of the application for treatment of a wound, a microbial infection and/or a dermatological disorder in a subject in need thereof. In another embodiment, the application provides methods for treatment of a wound, a microbial infection and/or a dermatological disorder in a subject in need thereof using a device or composition of the application. In a further embodiment, the application provides the use of a composition or device of the application for treatment of a wound, a microbial infection and/or a dermatological disorder in a subject in need thereof. In yet another embodiment, the application provides a composition or device of the application for use in the treatment of a wound, microbial infection and/or a dermatological disorder. In yet a further embodiment, the application provides the use of a composition of the application in the preparation of a medicament for the treatment of a wound, microbial infection and/or a dermatological disorder.

The term “treatment of a wound” as used herein means treatment or prevention of wounded tissue and includes, without limitation promoting at least one of the following results: decreased wound bacterial cell content, decreased size of wound, increased wound contraction by myofibroblasts, increased epithelialization by keratinocytes, increased cell migration, increased angiogenesis, increased fibroplasia, increased collagen deposition, increased fibronectin deposition, increased granulation tissue formation, and increased collagen remodeling.

The term “wound” as used herein refers to an injury wherein tissue, such as skin, is pierced, torn, cut or otherwise open and may involve skin, connective tissue, vessels, nerves, bone, joints, or organs. Types of wounds are known in the art and include without limitation, epithelial wounds. Briefly, venous stasis ulcers are due to the improper functioning of the veins in the legs. A diabetic foot ulcer is due to poor microcirculation in diabetics with high blood glucose and poor sensation. A sacral ulcer is an ulceration that occurs when lying immobilized in bed on the sacrum where increased pressure between the bed and skin compromises the local circulation. A trochanteric ulcer has the same etiology as a sacral ulcer but is on the pressure point of the hip (between bed and greater trochanter of the femur). An ischemic skin flap is poorly vascularized epithelialized soft tissue which will require time for vessels to grow into it through the process of angiogenesis or will become cyanotic and die due to lack of oxygenation. Normal wounds are defects of soft tissue due to injury (laceration, incision, abrasion, gun shot, etc) in which the epithelium is torn, cut, or punctured and can involve integument, epidermis, dermis, subcutaneous fat, blood vessels, nerves, muscle, even bone or organs. Chronic wounds are injuries that do not completely heal. Accordingly, in one embodiment, the wound is a chronic wound, a diabetic ulcer, a venous ulcer, a sacral ulcer, a gluteal ulcer, a trochanteric ulcer, a decubitus ulcer, a blister ulcer, a varicose leg ulcer, a finger ulcer, an ischemic skin flap, or a normal wound. In another embodiment, the wound is infected by bacteria or inflamed.

In one embodiment, the subject has a secondary condition, wherein the secondary condition, in the absence of treatments, delays wound healing or causes incomplete wound healing. Typical secondary conditions are diabetes, venous stasis, compromised circulation and irritation. In a particular embodiment, the secondary condition is diabetes.

In another embodiment, the wound is a result of a skin condition, including, without limitation, an inflammatory, autoimmune and infective skin condition.

The term “treatment of a microbial infection” as used herein means the treatment or prevention of microbial infected tissue and includes, without limitation, at least one of the following results: decreased microbial content; reduced inflammation; decreased white blood cell count; decreased fluid discharge; improved odor; improved blood flow and oxygenation.

The term “microbial infection” as used herein refers to an infection by a microorganism or a condition caused by a microorganism. In one embodiment, the microorganism is a bacterial, fungal, parasitic or viral microorganism and the infection is a bacterial, fungal, parasitic or viral infection. Bacterial infections include without limitation, infections caused by Gram-Negative Bacilli, Gram-Positive Bacilli, Gram-Positive Cocci, Neisseriaceae, and Mycobacteria.

Gram-Negative Bacilli include, without limitation, bartonella, brucellosis, campylobacter, cholera, E. coli, haemophilus, klebsiella, enterobacter, serratia, legionella, melioidosis, pertussis, plague, yersinia, proteeae, pseudomonas, salmonella, sigellosis, and tularemia. Gram-Positive Bacilli include without limitation organisms in anthrax, diphtheria, erysipelothricosis, Listeriosis, and nocardiosis. Gram-Positive Cocci include without limitation organisms of Pneumococcal, Staphylococcal, Streptococcal, and Enterococcal origin. Neisseriaceae include, without limitation, organisms of Acinetobacter, Kingella, Meningococcal, Moraxella catarrhalis, and Oligella origin. Mycobacteria include, without limitation, organisms of leprosy, tuberculosis, and mycobacteria resembling tubertulosis.

Parasitic infections include, without limitation, infections caused by protozoa selected from but not limited to the causative agents of: African Trypanosomiasis, Babesiosis, Chagas' Disease, Amebas, Leishmaniasis, Malaria, and Toxoplamosis.

Fungal infections include, without limitation, Tinea pedis, Onchyomycosis, Asperigillosis, Blastomycosis, Candidiasis, Coccidioidomycosis, Cryptococcosis, Histoplasmosis, opportunistic fungi, Mycetoma, Paracoccidioidomycosis, Pigmeted fungi, and Sporotrichosis.

Although little evidence exists in the literature, it is predicted that viruses from the families adenoviridae, picornaviridae, herpesviridae, hepadnaviridae, flaviviridae, retroviridae, togaviridae, rhabdoviridae, papillomaviridae, paramyxoviridae, and orthomyxoviridae should be susceptible to the antimicrobial properties of gNO due to the effects of NO on nucleic acids and the activity in NO in maintaining latency of infection. Accordingly, viral infections include, without limitation, infections caused by viruses of the families: Adenoviridae, Picornaviridae, Herpesviridae, Hepadnaviridae, Flaviviridae, Retroviridae, Togaviridae, Rhabdoviridae, Papillomaviridae, Paramyxoviridae, and Orthomyxoviridae.

The conditions caused by a microorganism include, without limitation, skin and soft tissue infections, bone and joint infections, surgical infections and hospital-acquired infections. These conditions may be persistent infections and/or intracellular infections. Such infections may be part of a wound, such as a chronic or surgical wound, or result in a dermatological disorder, as described herein.

In one embodiment, the microorganism causing the infection is drug resistant. In another embodiment, the microorganism is Vancomycin or Methacillin resistant.

The term “treatment of a dermatological disorder” as used herein means the treatment or prevention of tissue affected by a dermatological disorder and includes without limitation, at least one of the following results: reduction of a symptom of the disorder, elimination of a symptom of the disorder, alleviation of a symptom of the disorder, elimination of the source of the disorder.

Relaxation of vascular epithelial cells leads to an increase in capillary blood flow (Q) as described by Poiseuille's laws for laminar fluids. Increased arterial blood flow, increases the transport of nutrients to the tissues and increases the transport of metabolites away from tissues which can improve many factors that contribute to diseases of the skin. Improved oxygenation, more regulated pH, improved hydration of skin, increased access to mediators of immunity, and increased thickness of the vessel-containing dermal layer can all contribute to improvements in ongoing pathology. In the same way that NO acts to relax arteriolar vascular cells and increases blood flow, so to NO can vasodilate vascular smooth muscle leading to the promotion of vascular edema. Again, this process can allow for greater access to mediators of immunity.

Furthermore, by up regulation of iNOS, larger amounts of NO can be produced and act directly on microbial infections in mammals which are often causative agents in dermatologic disorders. Nitric oxide can, however, also indirectly support the eradication of microbial infections through modulation of the host immune response. Again, one of these ways is the modulation of the Th1 response and through modulation of cytokine levels. As many dermatologic disorders have an immune component to the pathophsyiology, these disorders can be treated by a regimen that provides exogenous nitric oxide for regulating the immune system.

Nitric oxide has also been found to be a signalling molecule for the recruitment of stem cells which can be used to replace lost components of dermis, epidermis, neural and vascular structures as well as provide the right extracellular matrix required for normal skin form and function and for normal repair.

As mentioned above, nitric oxide is a potent antimicrobial agent against bacteria, viruses, parasites, and fungus. As with many disorders, dermatologic disorders can have a pathogenesis that begins with an infection or the disorder may lead to infection. In the case of the former, an infectious agent can alter normal host cell activity, metabolism, or growth and cause the altered cell to differentiate (various cancers), change metabolism, or proliferate as is the case with verucca (warts).

Further, in light of the correlation between persistent NOS2 upregulation and inflammatory skin conditions such as Stevens-Johnson syndrome, it is quite conceivable that treatment with exogenous NO would be of benefit both through reduced recruitment of pro-inflammatory cells to the affected site and by re-establishing normal feedback inhibition of NOS expression.

In addition, barrier function impairment through dysregulation of NOS in dermatologic disorders, such as dermatitis, may also be reversed by use of exogenous NO to break the pathological dysregulation of NO. In addition, inhibition of oxidative damage is potentially beneficial in many dermatological disorders.

Accordingly, the dermatological disorder as used herein refers to a disturbance in the normal functioning of the skin and its appendages, such as hair and sweat glands and can be any dermatological disorder, including without limitation, acne, such as acne vulgaris, perioral dermatitis, rosacea, pruritus, urticaria, cellulitis, cutaneous abscess, erysipelas, erythrasma, folliculitis, furuncles and carbuncles, hidradenitis suppurativa, impetigo, eethyma, lymphadenitis, lymphangitis, benign tumors, dermatofibroma, epidermal cysts, keloids, keratoacanthoma, lipomas, atypical moles, seborrheic keratoses, vascular lesions, infantile hemangioma, nevus flammeus, port-wine stain, nevus araneus, pyogenic granuloma, lymphatic malformations, bullous diseases, bullous pemphigoid, dermatitis herpetiformis, epidermolysis bullosa acquisita, linear immunoglobulin A disease, pemphigus foliaceous, pemphigus vulgaris, cancers of the skin, basal cell carcinoma, Bowen's disease, Kaposi's sarcoma, melanoma, Paget's disease, squamous cell carcinoma, cornification disorders, corns, ichthyosis, xeroderma, keratosis pilaris, dermatitis of unknown origin, atopic dermatitis, contact dermatitis, exfoliative dermatitis, hand and foot dermatitis, lichen simplex chronicus, nummular dermatitis, seborrheic dermatitis, stasis dermatitis, dermatophytoses, dermatophytid reaction, intertrigo, tinea versicolor, alopecia, alopecia greata, hirsutism, pseudofolliculitis barbae, acute febrile neutrophilic dermatosis, erythema multiforme, erythema nodosum, granuloma annulare, panniculitis, pyoderma gangrenosum, Stevens-Johnson Syndrome (SJS), nail melanonychia striata, onychogryphosis, onycholysis, onychotillomania, trachyonychia, trauma, such as the discolouration left after bruising or trichohylane granules left behind after bruising, onychomycosis caused by infection, paronychia, chronic paronychia, lice, scabies, cutaneous larva migrans, autoimmune pigmentation disorders, vitiligo, pressure ulcers, ischemic and venous ulcers, scaling diseases, lichen planus, lichen sclerosus, parapsoriasis, pityriasis lichenoides, pityriasis rosea, pityriasis tubra pilaris, psoriasis, actinic keratoses, skin cancers, solar urticaria, polymorphous light eruption, bromhidrosis, hyperhidrosis, hypohidrosis, miliaria, molluscum contagiosum, warts, periungual refractory zoonotic diseases, contagious eethyma.

The term “subject” as used herein means an animal, optionally a mammal and typically a human.

In one aspect, the device or composition is kept inactive until the time of application of the device or composition onto the tissue, for example, by keeping the nitric oxide gas precursor and composition comprising the live cell, enzyme or catalyst separate, such as two creams or gels or by dehydrating the composition until use, such as with a powder composition or dissolvable film. Accordingly, in one embodiment, the application provides a method for treatment of a tissue of a wound, microbial infection and/or dermatological disorder in a subject in need thereof comprising:

contacting the tissue with a casing permeable to nitric oxide gas, the casing containing a plurality of inactive agents that, when activated, react to produce nitric oxide gas; and

activating the inactive agents to produce nitric oxide gas,

wherein the nitric oxide gas communicates through (i.e. passes through) the casing and contacts the tissue to treat the wound, microbial infection and/or dermatological disorder in the subject in need thereof.

The application also provides use of a casing permeable to nitric oxide gas for treating a wound, microbial infection and/or dermatological disorder, wherein the casing contains a plurality of inactive agents that, when activated, react to produce nitric oxide gas. The application further provides a casing permeable to nitric oxide gas for use in treating a wound, microbial infection and/or dermatological disorder, wherein the casing contains a plurality of inactive agents that, when activated, react to produce nitric oxide gas.

In another embodiment, the application provides a method for treating a wound, microbial infection or dermatological disorder in a subject in need thereof comprising providing inactive agents that, when activated, react to produce nitric oxide gas; activating the inactive agents to produce nitric oxide gas; and applying the activated agents to the tissue of the subject. The application also provides a use of inactive agents for treating a wound, microbial infection and/or dermatological disorder; wherein the inactive agents, when activated, react to produce nitric oxide gas. The application further provides inactive agents for use in treating a wound, microbial infection and/or dermatological disorder; wherein the inactive agents, when activated, react to produce nitric oxide gas. The application yet further provides a use of inactive agents for the preparation of a medicament for treating a wound, microbial infection and/or dermatological disorder; wherein the inactive agents, when activated, react to produce nitric oxide gas.

In one embodiment, the inactive agents comprise i) a nitric oxide gas precursor, and ii) (a) an isolated enzyme or a live cell expressing an endogenous enzyme, the enzyme having activity that converts the nitric oxide gas precursor to nitric oxide gas or having activity on a substrate that produces a catalyst that causes the conversion of the nitric oxide gas precursor to nitric oxide gas or (b) a live cell expressing a catalyst for converting the nitric oxide gas precursor to nitric oxide gas.

In another embodiment, the inactive agents comprise separated agents and activating the inactive agents comprise combining the separated agents. In one embodiment, the separated agents are combined by applying pressure or temperature to the device. In yet another embodiment, the inactive agents comprise dehydrated agents and activating the inactive agents comprise hydration.

In yet another embodiment, there is provided a method for treating a wound, microbial infection and/or dermatological disorder in a subject in need thereof comprising:

contacting the tissue with a nitric oxide gas releasing composition or device, the composition or device comprising an isolated enzyme or a live cell expressing an endogenous enzyme, the enzyme (i) having activity that converts nitrate to nitric oxide gas or (ii) having activity on a substrate that produces a catalyst that causes the conversion of nitrate to nitric oxide gas or (b) a live cell expressing a catalyst for converting nitrate to nitric oxide gas;

wherein the composition reacts with nitrate in sweat on the tissue to produce nitric oxide gas for treating a wound, microbial infection and/or dermatological disorder in the subject in need thereof.

In a further embodiment, the device or composition is applied to the tissue for a treatment period without inducing toxicity to the subject or tissue. The treatment period will depend on the type of device or composition used. For example, for a device described herein, the treatment period typically is from about 1 to 24 hours, preferably about 6-10 hours and more preferably about 8 hours. For a cream composition, the cream is typically applied one to three times daily. For a mask composition, the treatment period is typically from about 1 to 8 hours, optionally, 1-2 hours.

In yet a further embodiment, the NO is produced by the device or composition in an amount suitable for the particular use and can range from 1 to 1000 parts per million volume (ppmv). In one embodiment, the NO produced by the device or composition for wounds is from about 1 to 1000 ppmv. In another embodiment, the NO produced by the device or composition for infections is from about 150 to 1000 ppmv. In yet another embodiment, the NO produced by the device or composition for dermatological disorders is from about 5 to 500 ppmv.

A two-step application of nitric oxide, the first with a high concentration, and the second with a low concentration, is known to promote wound healing. Accordingly, in another aspect, the application provides a method to promote healing of a wound in a subject in need thereof comprising:

first exposing the wound to a device of the application to produce a high concentration of nitric oxide gas/radical that contacts the wound for a first treatment period; and

second exposing the wound to a second device of the application to produce a low concentration of nitric oxide gas/radical that contacts the wound for a second treatment period. A high concentration of nitric oxide gas is from about 100 to 400 ppm and a low concentration of nitric oxide gas is from about 1 ppm to 50 ppm. In one embodiment, the high concentration is about 200 ppm. In another embodiment, the low concentration is about 5 ppm.

Nitric oxide is also used in the meat industry in improving red meat products. Accordingly, in one embodiment, the application provides use of a device of the application for improving red meat product shelf life, preservation, or physical appearance. The method of use of the device involves exposing the red meat product to the device so that NO contacts the red meat product. In a particular embodiment, the improved appearance comprises improved colour with increased redness and reduced brown, green, black, or iridescent colour. In another embodiment, the nitric oxide inhibits oxidative processes in the meat.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Results

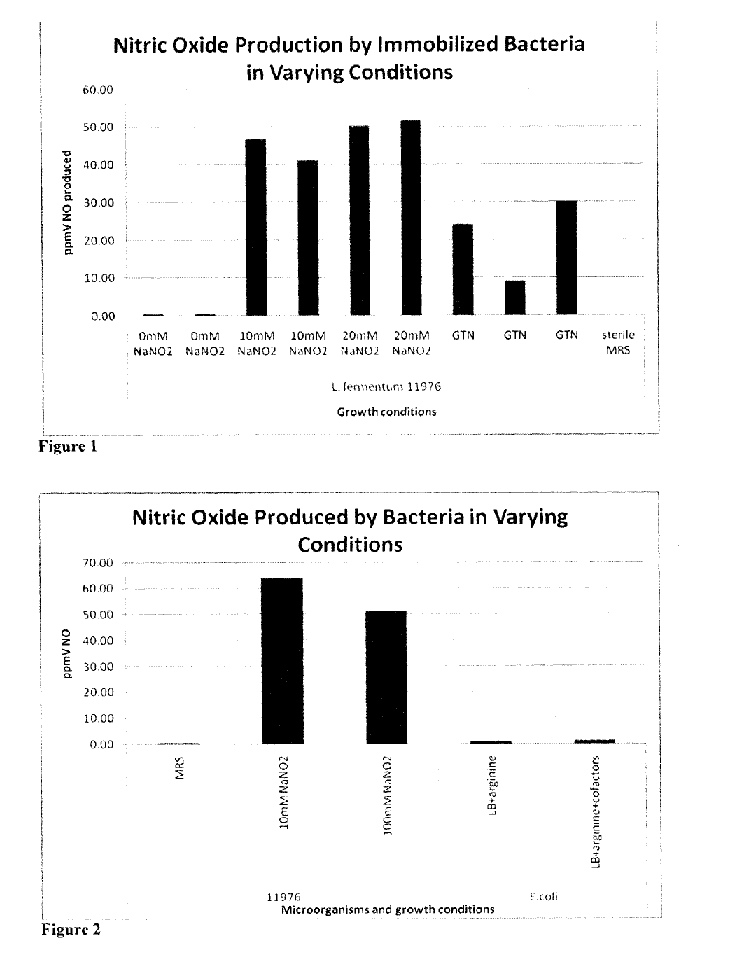

Tables 2-4 show the reaction that produces nitric oxide from a precursor. The results also show that live bacteria are able to produce nitric oxide gas (gNO) when immobilized in a slab-like piece of agarose supplemented with MRS growth media and either nitrite or a nitroglycerine patch (FIG. 1). The results in FIG. 2 show that live bacteria are able to produce nitric oxide gas when grown in media with the indicated cofactors. Without wishing to be bound by theory, the most probable mechanism for nitric oxide production from nitrite is the reduction of the salt to gNO by lactic acid produced by the metabolically active bacteria. The most probable mechanism of gNO production from nitroglycerine is that the organisms produce lactic acid which reduces nitroglycerine to nitrite and the resulting nitrite is reduced to nitric oxide again by lactic acid. In this way, the immobilized bacteria are capable of releasing gNO from a medical device or composition and onto affected tissue, over a period of time and in proportion to their metabolic activity.

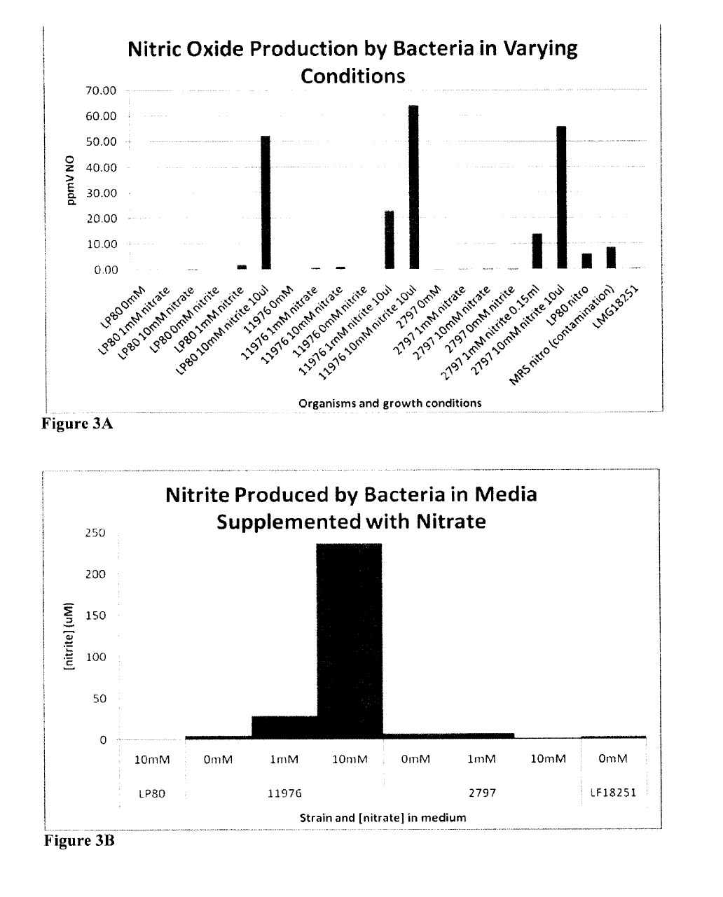

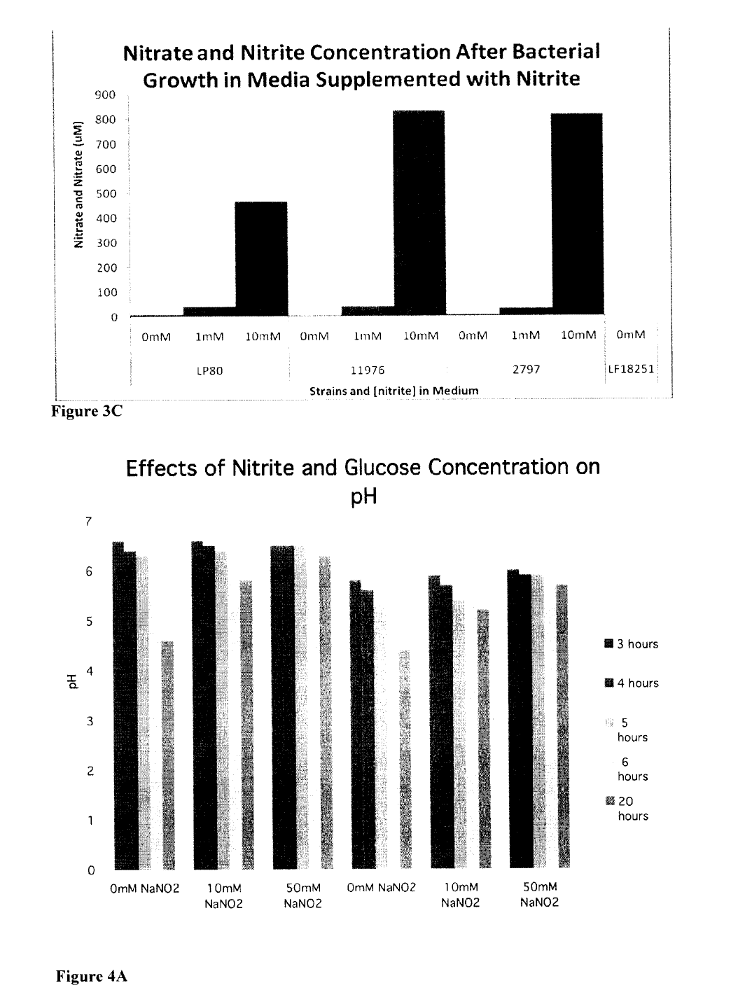

Nitrite salts can be reduced to gNO by several different lactic acid producing bacteria (LAB) and the quantity of gNO produced depends on the concentration of nitrite substrate and the acid producing capability of the bacteria (FIG. 3A). Some bacteria such as Lactobacillus fermentum (ATCC 11976) have a nitrate reducing capacity and hence nitrates, such as potassium nitrate, can be used as substrate for the production of gNO by these bacteria. The nitrate substrate can be converted to nitrite which can then be reduced to gNO by lactic acid produced by the bacteria (FIG. 3B). Again, this example substantiates the use of nitrates, nitrites, or some other nitric oxide donator as a substrate with live cells or enzymes in a medical device or composition for treating affected tissue.

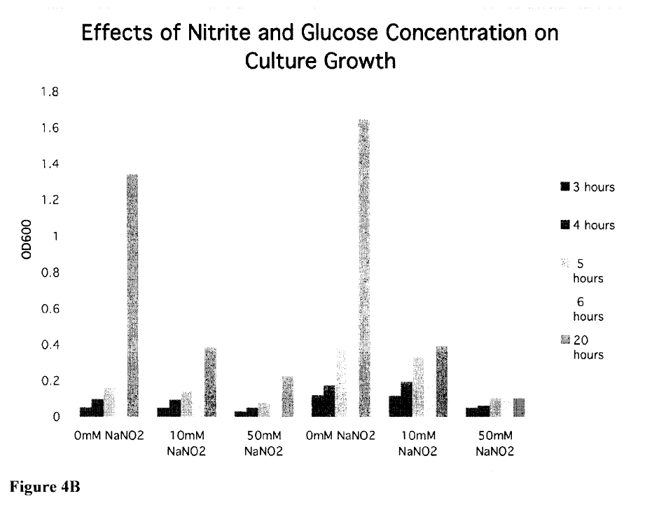

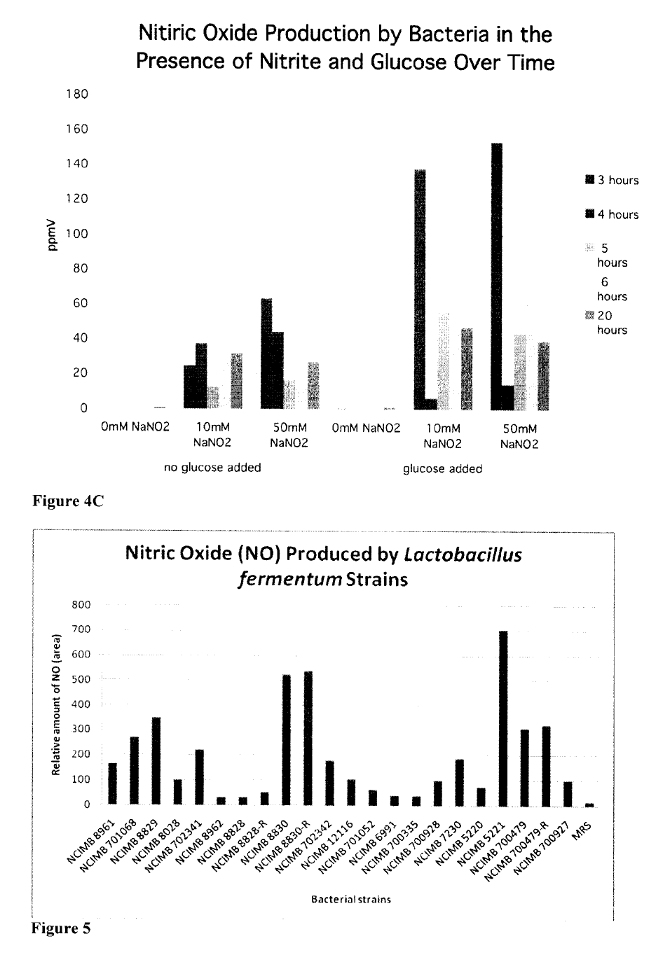

The addition of glucose to growth media containing LAB results in increased acidification of the growth media over time (lower pH). When supplemented with glucose, lower pH values were achieved with Lactobacillus fermentum (ATCC 11976) over time (FIG. 4A). The addition of nitrite to the growth media, although making more substrate available for the production of gNO, inhibited the growth of bacteria as seen by reduced OD600 values (FIG. 4B). Increased concentrations of lactic acid (lower pH values) were observed in media supplemented with glucose and despite the inhibition of bacterial growth at higher concentrations of nitrite, an increased capacity for reduction and more gNO was produced by bacteria in growth media supplemented with both glucose and nitrite (FIG. 4C). A pattern of increasing and decreasing gNO concentrations was seen. The interplay between LAB, growth media, glucose, NO substrate, NO, and lactic acid provides a useful therapeutic system for treating wounds, microbial infections and/or dermatological disorders. The continued release of gNO by immobilized or microencapsulated live cells or enzymes over the entire therapeutic duration is very advantageous for this cell/enzyme based technology.

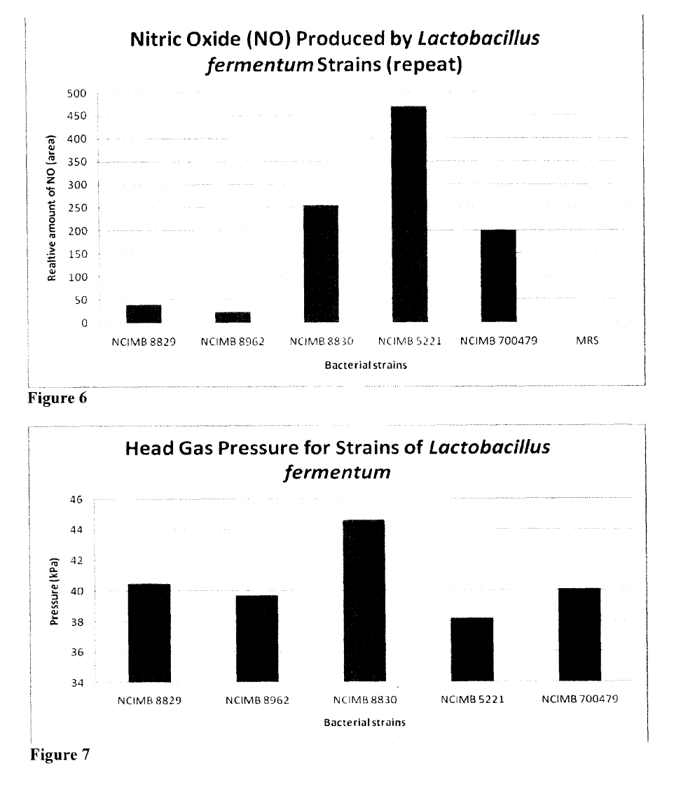

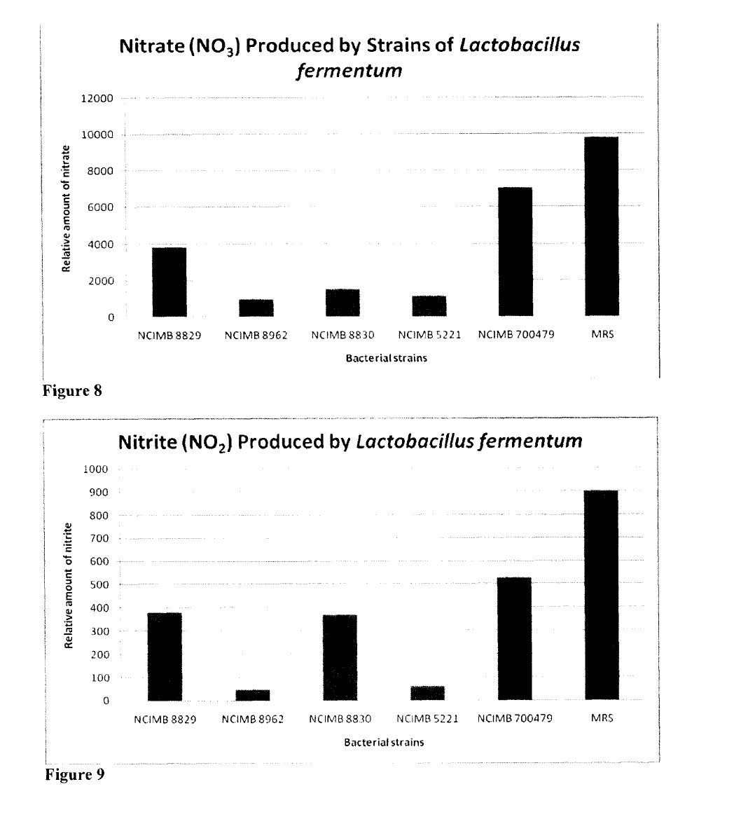

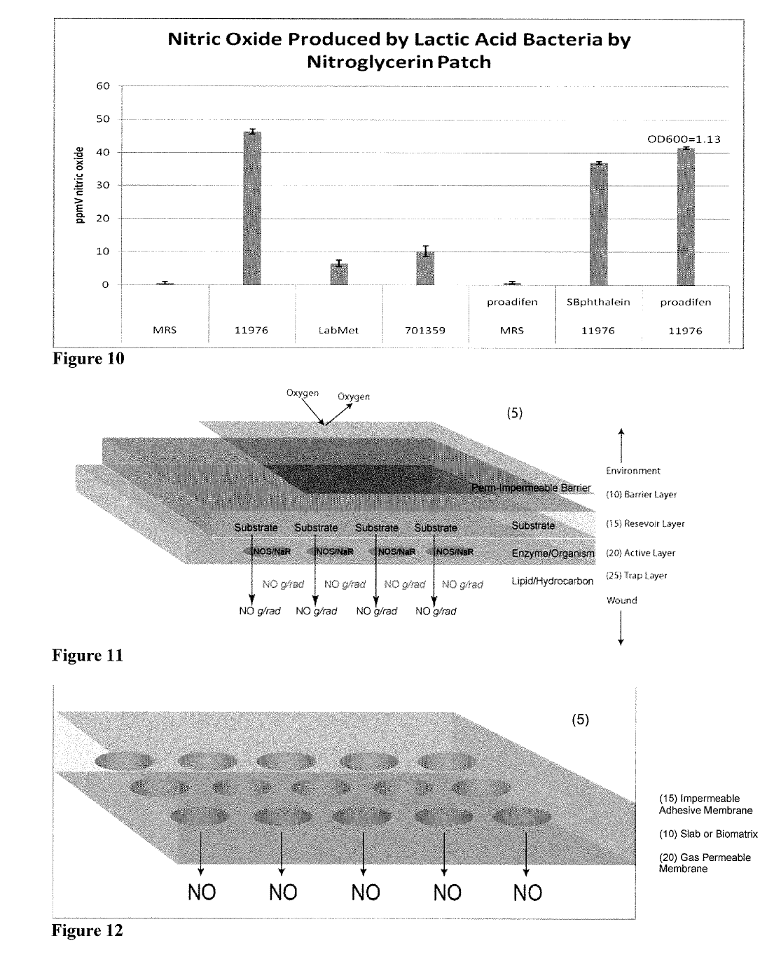

The results also show that some strains of Lactobacillus are capable of producing nitric oxide when grown in MRS broth (FIG. 5 and FIG. 6). The head gas pressure was also measured in the vessel where the bacterial strains were grown (FIG. 7). The present inventors have also shown the ability of the bacterial strains to produce nitrate and nitrite after growth in media for 20 hours (FIGS. 8 and 9). Nitric oxide is also produced from lactic acid bacteria by a use of a nitroglycerin patch (FIG. 10).

FIGS. 11-14 provide examples of devices that are used to provide a source of nitric oxide to affected tissue.

FIG. 11 shows a multilayered nitric oxide producing medical device (5) made up of a barrier (10), reservoir (15), active (20), and trap layer (25) as one proceeds from the environment to the affected tissue. The barrier layer (10) maintains variable permeability to oxygen while protecting the affected tissue and adhering the patch. The reservoir layer (15) contains substrate, such as potassium nitrite or arginine, for the enzyme in the active layer. The active layer (20) contains enzyme producing microorganisms or free enzyme and cofactors for the production of nitric oxide. The trap layer (25) is made up of lipids or hydrocarbons for concentrating nitric oxide radicals nearest the affected tissue.

FIG. 12 shows a single layered device (5) with NO producing bacteria immobilized in polymer slab or biomatrix (10) for the production of NO for the treatment of wounds, microbial infections and/or dermatological disorders. The production of NO is maintained by the immobilized cells and protected from contact with O₂ by an impermeable adhesive membrane (15) above the immobilized bacteria. Also, the transmission of other biologic material can be prevented from coming into contact with the affected tissue by a gas permeable membrane (20).

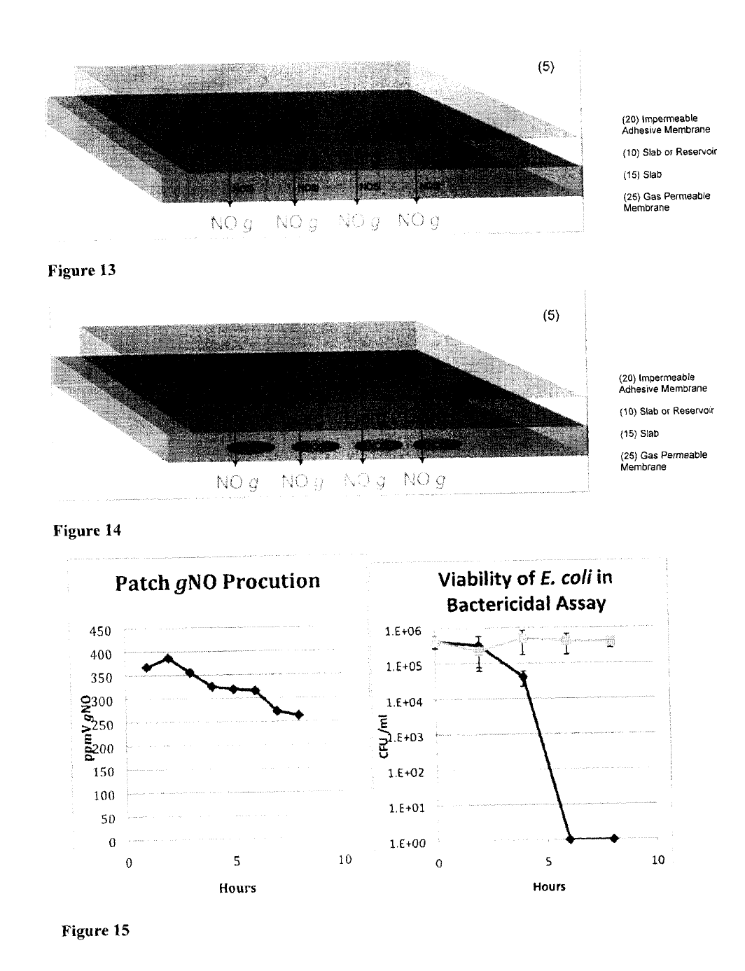

FIG. 13 shows a simple layered medical device (5) with L-arginine immobilized in slab or in a reservoir (10) above NOS enzyme immobilized in a slab (15) for the production of NO for the treatment of wounds, microbial infections and/or dermatological disorders. The production of NO is maintained by the immobilized cells and protected from contact with O₂ by an impermeable adhesive membrane (20) above the immobilized bacteria. Also, the transmission of other biologic material can be prevented from coming into contact with the affected tissue by a gas permeable membrane (25).

FIG. 14 shows a simple layered medical device (5) with L-arginine immobilized in slab or in a reservoir (10) above NOS producing bacteria immobilized in an alginate slab (15) for the production of NO for the treatment of wounds, microbial infections and/or dermatological disorders. The production of NO is maintained by the immobilized cells and protected from contact with O₂ by an impermeable adhesive membrane (20) above the immobilized bacteria. Also, the transmission of other biologic material can be prevented from coming into contact with the affected tissue by a gas permeable membrane (25).

Live Cell or Enzyme Having Activity that Produces a Catalyst

A crude extract of pancreatic enzyme (5% pancreatin) is optionally immobilized in a slow gelling hydropolymer of alginate (2% alginic acid, sodium pyrophosphate, calcium sulphate, water) with a protein/lipid containing substrate (1% soy protein isolate) and a nitric oxide donor salt (NaNO₂). Alternatively, a reducing agent such as sodium iodide (NaI) is optionally used to improve the stoichiometry of the reaction and provide the added bactericidal effects of iodine gas. This device or patch is typically lyophilized and stored for later use. Once made active by the addition of water and with a gas impermeable and optionally adhesive backing and a gas permeable but protective tissue interface (or contact surface), is useful to produce high or low therapeutic levels of nitric oxide gas. The NO gas is useful in therapy including, without limitation, topical clinical therapy of wounds, dermatological disorders, degenerative disease and certain surgical applications. Such uses include, without limitation, use as an anti-microbial agent, scar formation inhibitor, in chronic wound healing, for improved surgical flap survival by vasodilatation.

### Materials and Methods:

### NO Gas Production by Immobilized Bacteria in Varying Conditions (FIG. 1)

MRS agar (Fisher scientific) was autoclaved in a Wheaton bottle (Fisher scientific) capped with a septum-equipped PTFE cap. Once the agar was cooled, but still liquid, sodium nitrite (Sigma-Aldrich) was added to the desired final concentration from a sterile 1M stock. Alternatively, a Nitro-Dur 0.8 transdermal nitro-glycerine patch (Key pharmaceuticals) was introduced in the bottle. An overnight culture of Lactobacillus fermentum (ATCC 11976) (OD600=2) was used to aseptically inoculate the agar to a 1:50 dilution. The agar was left to harden at room temperature for 30 minutes and then incubated for 20 hours at 37° C. A 100 μL syringe (Hamilton) was used to remove gas from the headspace and to inject it in the injection port of a chemiluminescence NO analyzer (Sievers®, GE analytical). The area under the curve for each injection was recorded and the parts per million by volume value was calculated using a pre-determined conversion factor.

Growth of Lactobacillus fermentum (ATCC 11976) (FIG. 2)

MRS broth (Fisher scientific) was autoclaved in a Wheaton bottle (Fisher scientific) capped with a septum-equipped PTFE cap. Sodium nitrite (Sigma-Aldrich) was added to the desired final concentration from a sterile 1M stock. An overnight culture of Lactobacillus fermentum (ATCC 11976) (OD600=2) was used to aseptically inoculate the broth to a 1:50 dilution. After 20 hours at 37° C., a 100 μL syringe (Hamilton) was used to remove gas from the headspace and to inject it in the injection port of a chemiluminescence NO analyzer (Sievers®, GE analytical). The area under the curve for each injection was recorded and the parts per million by volume value was calculated using a pre-determined conversion factor.

Growth of Escherichia coli BL21 (pnNOS) (pGroESL) (FIG. 2)

An E. coli strain harboring a plasmid encoding the rat neuronal nitric oxide synthase (pnNOS) and a plasmid encoding chaperone proteins (pGroESL) was grown for 20 hours in LB containing 100 μg/ml ampicillin and 10 μg/ml chloramphenicol. 1 mM arginine was added and the cofactors required for neuronal nitric oxide synthase activity (12 μM BH4, 120 μM DTT and 0.1 mM NADPH) were added to one of the cultures. Sampling of the head gas was done as described above.

### Nitric Oxide Production by Bacteria in Varying Conditions (FIG. 3)

MRS broth (Fisher scientific) was autoclaved in a Wheaton bottle (Fisher scientific) capped with a septum-equipped PTFE cap. Sodium nitrite (Sigma-Aldrich) was added to the desired final concentration from a sterile 1M stock. An overnight culture of Lactobacillus fermentum (ATCC 11976), Lactobacillus plantarum LP80, Lactobacillus fermentum NCIMB 2797 or Lactobacillus fermentum (LMG 18251) (OD600=2) was used to aseptically inoculate the broth to a 1:50 dilution. After 20 hours at 37° C., a 100 μL syringe (Hamilton) was used to remove gas from the headspace and to inject it in the injection port of a chemiluminescence NO analyzer (Sievers®, GE analytical). The area under the curve for each injection was recorded and the parts per million by volume value was calculated using a pre-determined conversion factor.

### Nitrite Measurements (FIG. 3)

Nitrite levels were measured by injecting 1 ml of the growth medium in the reaction vessel of the chemiluminescence NO analyzer (Sievers®, GE analytical) containing 3 ml glacial acetic acid and 1 ml 50 mM KI. Reaction of the nitrite with the acid and the KI releases NO gas which is in turn detected by the analyzer.

### Nitrate Measurements (FIG. 3)

Nitrate levels were measured by injecting 1 ml of the growth medium into the reaction vessel of the chemiluminescence NO analyzer (Sievers®, GE analytical) containing 3 ml 1M HCl and 50 mM VCI₃. The reaction was performed at 95° C. using the heating water bath and pump to heat the reaction vessel to 95° C. Reaction of the nitrate in the sample with the acid and the VCI₃ releases NO gas which is in turn detected by the analyzer.

### Nitric Oxide Production by Bacteria in the Presence of Nitrite and Glucose Over Time (FIG. 4)

MRS broth (Fisher scientific) with the required amount of glucose (20 g/L or 100 g/L) was autoclaved in a Wheaton bottle (Fisher scientific) capped with a septum-equipped PTFE cap. Sodium nitrite (Sigma-Aldrich) was added to the desired final concentration from a sterile 1M stock. An overnight culture of Lactobacillus fermentum 11976 (OD600=2) was used to aseptically inoculate the broth to a 1:50 dilution. After growth at 37° C. for the required amount of time without shaking, a 1 ml syringe equipped with a 27G 1.25″ needle was used to puncture the septum and remove 0.7 ml of the medium. This aliquot was used to perform pH (FIG. 4A) and spectrophotometric (FIG. 4B) measurements. The septum was then punctured with a 100 μL syringe (Hamilton) to remove gas from the headspace and injected in the injection port of a chemiluminescence NO analyzer (Sievers®, GE analytical). The area under the curve for each injection was recorded and the parts per million by volume value was calculated using a pre-determined conversion factor (FIG. 4C).

Nitric Oxide Production by Lactobacillus fermentum (FIGS. 5-9)

Strains of Lactobacillus fermentum (NCIMB, Scotland) were grown for 20 hours in a septum-equipped bottle containing 20 ml of MRS broth. The pressure in the bottle resulting from gas production was measured using a manometer (Fisher scientific) equipped with a needle to puncture the septum. 1 ml of head gas was withdrawn and injected in a nitric oxide analyzer (Seivers, General Electric) and the area under the curve was reported as a representation of the relative amount of nitric oxide gas present in the headspace. 10 ul of the medium was subsequently withdrawn and injected in the analyzer with glacial acetic acid and excess sodium iodide present in the injection chamber. This resulted in the nitrite being converted to nitric oxide gas which is then measured by the analyzer and reported as the relative amount of nitrite in the growth medium. The same process was repeated for the measurement of nitrate in the growth medium except that 1M HCl and excess vanadium chloride was present in the injection chamber to convert the nitrate in the medium to nitric oxide gas. The gas thereby measured by the analyzer gave a relative measure of the amount of nitrate in the growth medium.

### Nitric Oxide Produced by Lactic Acid Bacteria by Nitroglycerin Patch (FIG. 10)

MRS agar (Fisher scientific) was autoclaved in a Wheaton bottle (Fisher scientific) capped with a septum-equipped PTFE cap. Once the agar was cooled, but still liquid, a Nitro-Dur 0.8 transdermal nitroglycerin patch (Key pharmaceuticals) was introduced in the bottle. An overnight culture of Lactobacillus reuteri (NCIMB 701359), Lactobacillus reuteri (LabMet) or Lactobacillus fermentum (ATCC 11976) (OD600=2) was used to aseptically inoculate the agar to a 1:50 dilution. Proadifen (SKS-525A), an inhibitor of the P450 enzyme was added to a final concentration of 50 μM from a 64 mM stock in water and sulfobromophthalein, an inhibitor of gluthathione-S-transferase, was added to a concentration of 1 mM from a 30 mM stock in water. The agar was left to harden at room temperature for 30 minutes and then incubated for 20 hours at 37° C. A 100 μL syringe (Hamilton) was used to remove gas from the headspace and to inject it in the injection port of a Sievers NO analyzer (GE analytical). The area under the curve for each injection was integrated and recorded and the parts per million by volume value was calculated using a pre-determined conversion factor.

### Example 2

### Results

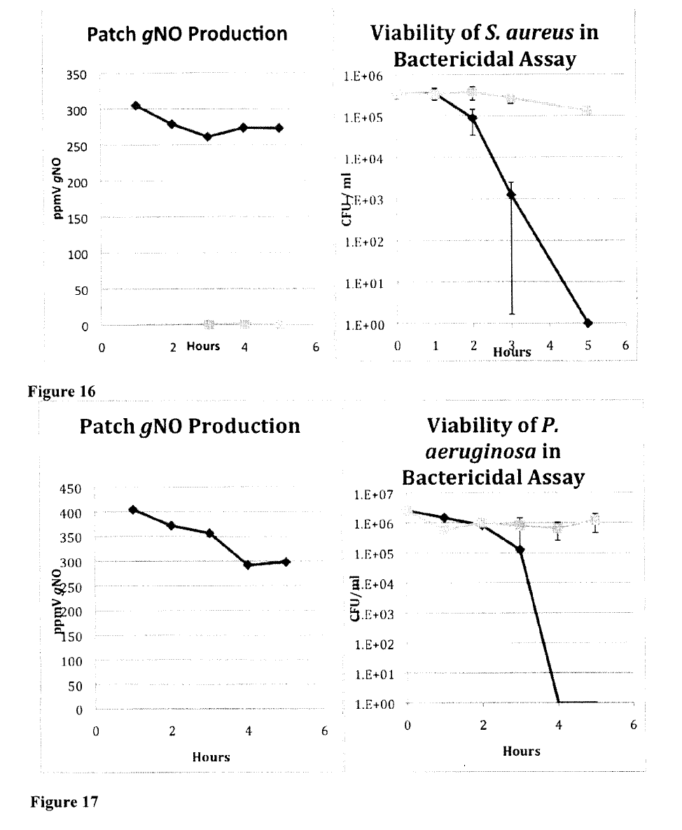

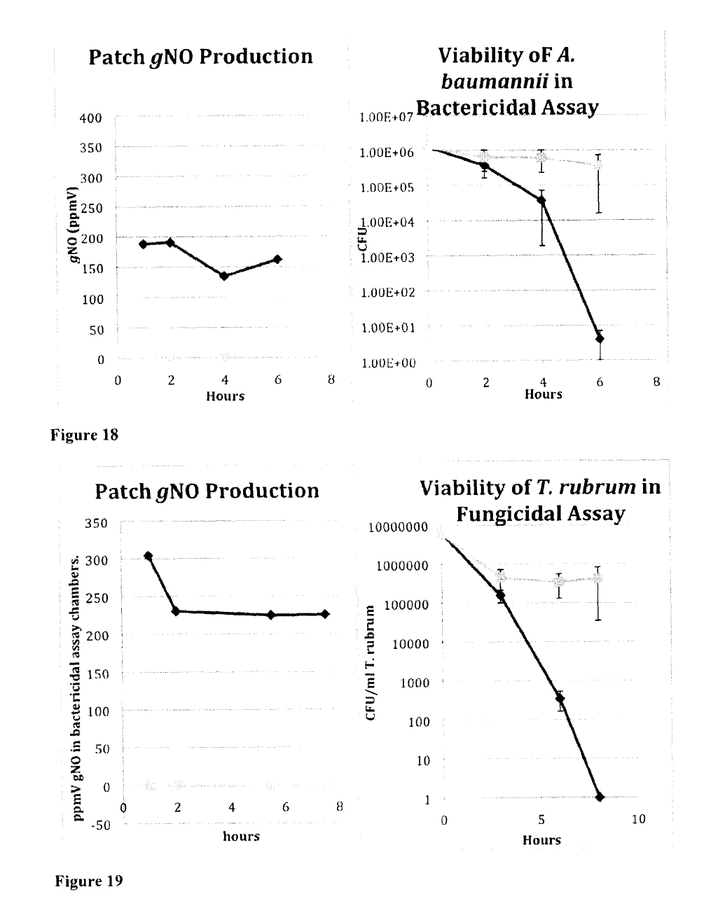

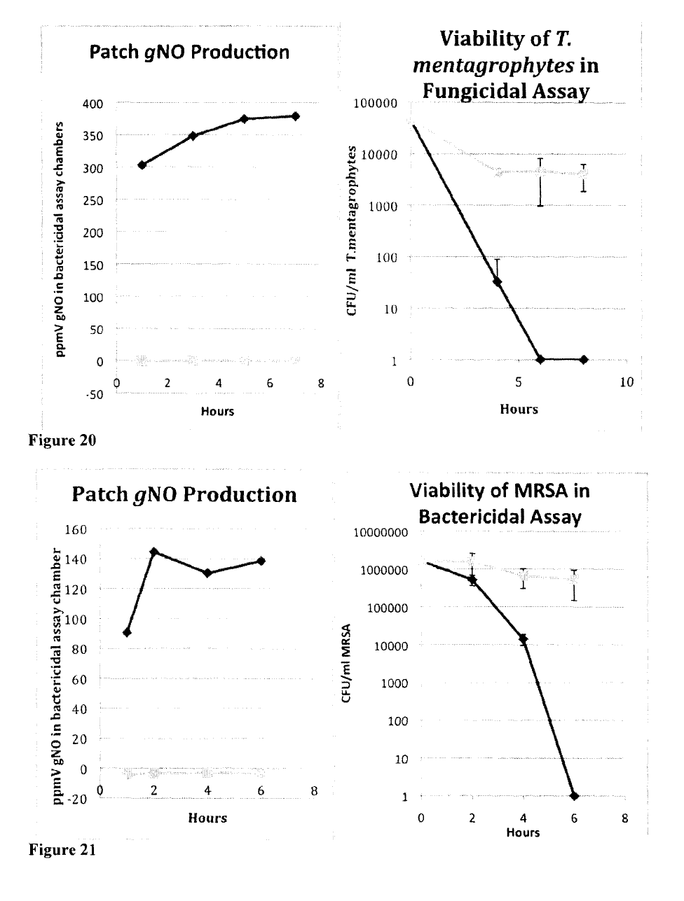

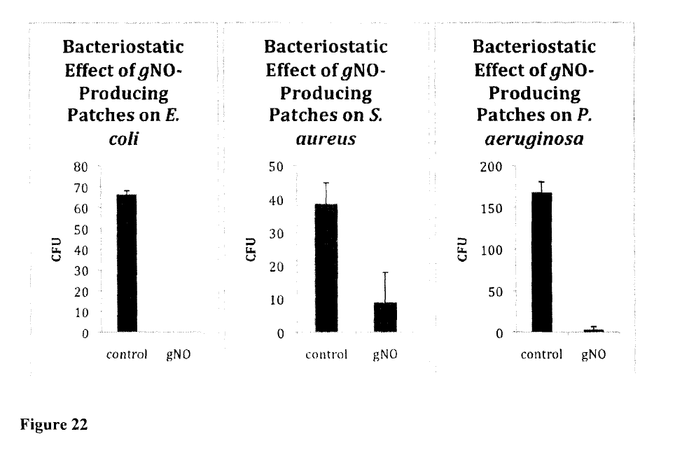

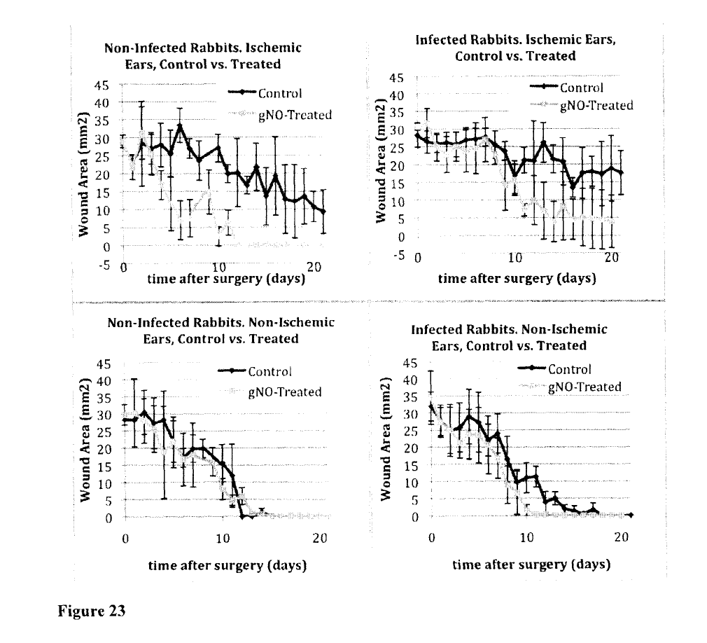

The gNO-producing patches showed a bactericidal effect on E. coli (FIG. 15), S. aureus (FIG. 16), P. aeruginosa (FIG. 17), A. baumannii (FIG. 18), and MRSA (FIG. 21). The gNO-producing patches showed a fungicidal effect on T. rubrum (FIG. 19) and T. mentagrophytes (FIG. 20). The gNO-producing patches also showed bacteriostatic effects on E. coli (FIG. 22 (left)), S. aureus (FIG. 22 (middle)), and P. aeruginosa (FIG. 22 (right)).

### Materials and Methods

Patch Preparation: A one-sided gas permeable pocket was created by heat sealing 3 sides of a rectangular gas permeable membrane (Tegaderm) with a heat sealable plastic film. The resulting pocket was filled up with an alginate-immobilized L. Fermentum wafer and a glucose/NaNO₂ solution and the fourth side of the pocket was heat sealed. A layer of aluminized tape was applied to the plastic film to avoid loss of gas. Control patches are made with a glucose solution that does not contain the NO donor NaNO₂.

Bactericidal Assay: Assay chambers that consist of a 6 ml cylindrical cavity containing liquid and gas sampling ports were designed specifically to test the bactericidal effect of gNO-producing patches. The chambers were filled with 3 mls of bacterial suspensions in saline (approximately 10⁵ CFU/ml) and were sealed with a control or gNO-producing patch. Liquid samples were obtained every 2 hours from the liquid port and serial dilutions were plated on growth medium/agar. Colonies were counted after an overnight incubation at 37 C.

gNO Measurements: A known volume of gas was sampled every hour with a Hamilton syringe from the gas port of the assay chamber and gNO content was measured with a chemiluminescence analyzer (Sievers).

Bacteriostatic Assay: Petri dishes filled with growth medium/agar broth were plated with approximately 30-to-100 colony CFU of bacteria and a gNO-producing patch, or control patch was placed on the dish lid. The dishes were sealed and placed upside-down in a 37° C. incubator, overnight. Colonies were counted the following day.

### Example 3

### Pilot Pre-Clinical Study

A pilot study was performed to provide information on the ability of nitric oxide to improve wound healing. The model uses the ischemic ear model in the rabbit, a well-validated model of ischemic wounds. Establishing ischemia involves a minor surgical procedure on the ear and the healing characteristics are similar to human healing in that it requires the generation of granulation tissue and reepithelization.

### Results

This pilot study provided very promising data on the efficacy and safety of the nitric oxide producing dressing. It was found that treated ischemic wounds healed faster than controls and that improvements could also be seen in the histological evaluation of the wounds.

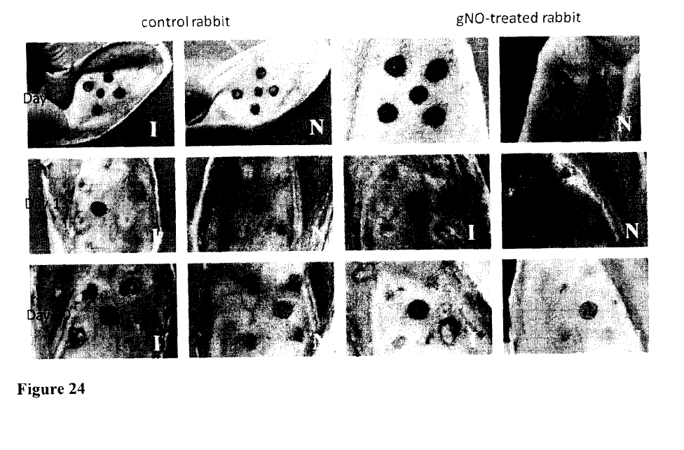

It was found that non-ischemic wounds closed between 10 and 15 days post-surgery, whether infected or not. The treatment of non-ischemic wounds with gNO marginally accelerated healing, as compared to the vehicle control (see FIG. 23, lower panels). Furthermore, the treatment of ischemic wounds with gNO resulted in visible improvement in the closure of both infected and non-infected wounds as compared to the vehicle control treated wounds (see FIG. 23, upper panels). All non-infected ischemic gNO-treated wounds were closed by day 15 post-surgery while 75% of gNO-treated infected ischemic wounds were closed by day 20 (FIG. 23). In contrast, vehicle control treated ischemic wounds showed poor healing overall, with a worsening observed in the infected wounds (FIG. 24).

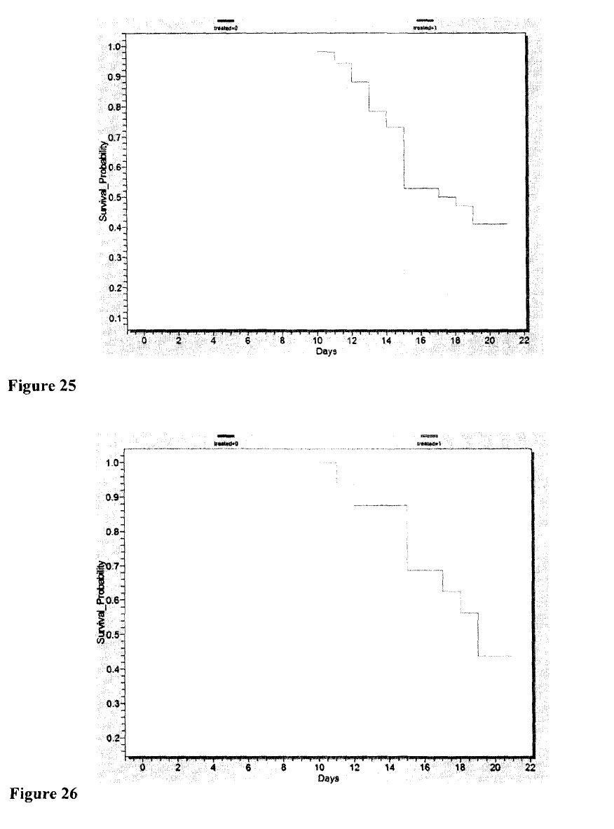

Kaplan-meier curves, also called survival curves, express the likelihood of survival over time and were used to represent the likelihood of wound closure over time. The data was plotted using time to closure of each wound separately, on a Kaplan-meier graph and statistical analysis was performed using two variables present in the pilot study: Time to closure and treatment. A significant reduction in the hazard ratio was observed for the treated group vs the non-treated, indicating that treated wounds were significantly more likely to heal than non-treated wounds. Kaplan-meier plots and Cox proportional hazard regression plots of the data were plotted and are presented in FIGS. 25 and 26 and Tables 7 and 8. Statistical analysis shows a significant improvement in time to closure of the treated group.

Histological evaluation of the ischemic wounds on the ears of rabbits treated with a vehicle control or with an gNO producing wound dressing was performed (Table 5). The results show an overall trend towards improvement in the healing of the wounds, both in an increase in the maturity and in a reduction of hyperplasia, crusting/exudates present and lowered inflammation/infiltration. The results are not statistically significant due to the small size of the study groups (2 ischemic ears treated with NO and 2 ischemic ears treated with vehicle control).

Toxicology data was collected and is summarized in Table 6. Direct observation of the rabbits did not yield any signs of overt toxicity to the gNO as the animals were generally healthy and did not show signs of distress related to the gNO producing dressing. No significant changes were observed between treated and vehicle control animals. Weight loss was measured at the end of the 21-day treatment period. Blood morphophology and hematology were performed by an external laboratory. Hematological analysis was performed with an ADVIA 120 analyser. The following parameters were evaluated: Red blood cell counts, haemoglobin, hematocrit, mean corpuscular volume (MCV), mean corpuscular haemoglobin (MCH), mean corpuscular haemoglobin concentration (MCHC), platelet count, white blood cells (WBC), WBC differential counts, cell morphology, and reticulocyte count. Blood smears were also prepared to evaluate morphology. Blood chemistry was performed internally on a Hitachi 911 analyser. Methemoglobine quantification was performed according to a modified Evelyn-Mallow method (Hegesh et al, 1970).

### Materials and Methods

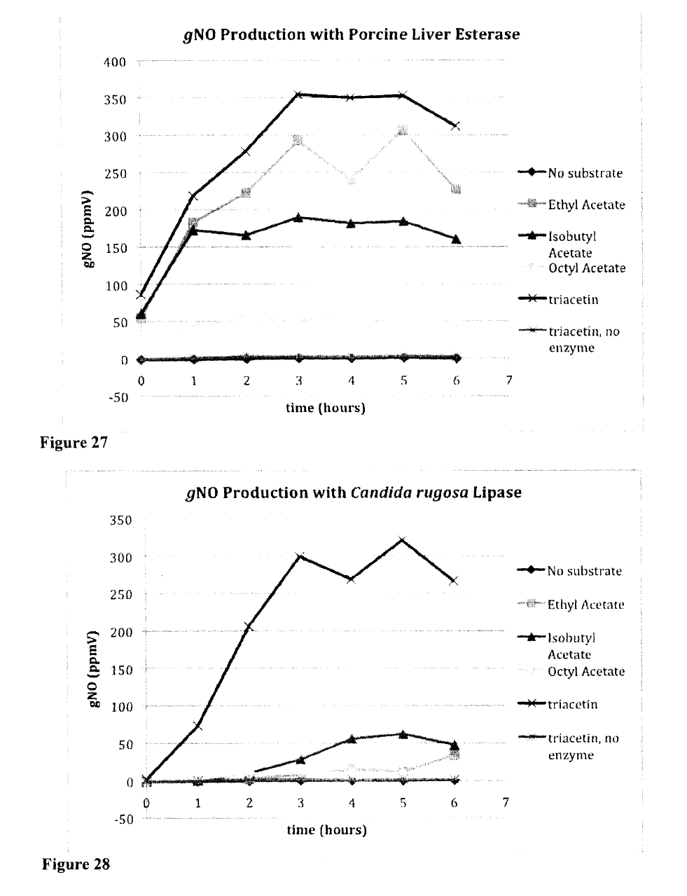

Preclinical Study Design: The effects of gNO-producing devices were compared to vehicle controls in 4 different experimental conditions: a) ischemic non-infected wounds, b) ischemic infected wounds, c) non-ischemic non-infected wounds, and d) non-ischemic infected wounds. A photographic summary of the evolution of infected wound healing is presented in FIG. 27.

Histopathological Evaluation: Tissue samples were left to fix for at least 24 hours in formalin, samples were bisected, placed in cassettes and processed to paraffin, and sections were sectioned at approximately 5 μm, mounted on glass slides and stained with hematoxylin and eosin (H&E) and Masson's trichrome stains. Fixation, mounting, staining and analysis of the stained samples were performed by AccelLAB Inc. pathologist using a semi-quantitative grading system.

Toxicologic Evaluation: Toxicity of gNO treatment was assessed for each of the four rabbits. Toxicology information was collected during and after the trial. Hematological evaluation and blood morphology was performed by an external lab while the blood chemistry was performed using a Hitachi 911 blood analyser.

### Example 4

### Generation of gNO Using Enzyme (Esters, Esterases, or Lipases) and NaNO₃

The hydrolysis of either esters or triglycerides results in the production of acids and alcohol. Herein, it is proposed the use of the hydrolysis of esters to generate acid sustainably for up to 48 hours in order to catalyze the dismutation of an NO donor, optionally nitrite, and release at least 200 ppmV of gNO during the indicated period of time. Among the enzymes that catalyze the hydrolysis of esters, there is a distinction between esterases and lipases depending on the substrate preferences. Whereas esterases have higher affinities for esters of low molecular weight, lipases recognize mainly triglycerides of fatty acids although the specificity of each enzyme may vary considerably.

### Materials and Methods

Enzymatic Generation of gNO: A 200 μl reaction solution was prepared by combining water, an acetate ester (ethyl acetate, isobutyl acetate, octyl acetate) or a triglyceride such as triacetin (glyceryl triacetate), sodium nitrite, and an esterase (porcine liver esterase, rhyzopus oryzae esterase) or a lipase (porcine pancreatic lipase, candida rugosa lipase). The solution was then added to a 2 ml vial, which was closed tightly with a septum cap. The head gas was sampled every hour from the reaction containing vials in order to determine gNO concentrations.

Patch Preparation: A one-sided gas permeable pocket was created by heat-sealing 3 sides of a rectangular gas permeable membrane (Tegaderm) with a heat sealable plastic film. The resulting pocket was filled up with a triacetin/candida rugosa lipase/NaNO₂ solution and the fourth side of the pocket was then heat-sealed. A layer of aluminized tape was applied to the plastic film to avoid loss of gas. Lyophilised alginate microbeads were added to the solution in some patches to improve the consistency or physical properties of the device.

gNO Measurements: A known volume of gas was sampled hourly from the gas port of the assay chamber with a Hamilton syringe and gNO content was measured with a chemiluminescence analyzer (Sievers).

### Results and Discussion

A number of enzymes are available for the hydrolysis of ester bonds. The advantage of utilizing the hydrolysis of esters or triglycerides is the reaction results in relatively innocuous by-products and weak acids. Using the right enzyme, with the right substrate, allows for the production of a nitric oxide producing dressing with minimal risk of toxicity. Work was performed to determine which enzymes could be used as well as the best possible substrate. FIG. 27 presents the results of experiments using porcine liver esterase against 4 substrates: Ethyl acetate, Isobutyl acetate, octyl acetate and triacetin. All 4 substrates produce acid upon hydrolysis by the enzyme, leading to nitric oxide production. Three of the substrates led to biologically relevant production of nitric oxide, reaching 200 ppmV in 1 hour. Triacetin was the strongest acid producer after hydrolysis, leading to upward of 350 ppmV over the 6 hour experiment.

Candida rugosa lipase is another enzyme able to hydrolyse ester bonds, though limited to triglyceride substrates. The enzyme was tested against four substrates and it was found that only triacetin, a simple triglyceride, was able to produce high amounts of nitric oxide (FIG. 28). The hydrolysis of triacetin by esterase or lipase leads to the production of glycerol and acetic acid, both innocuous compounds acceptable in a wound healing dressing or a dressing for treating a microbial infection or dermatological disorder.

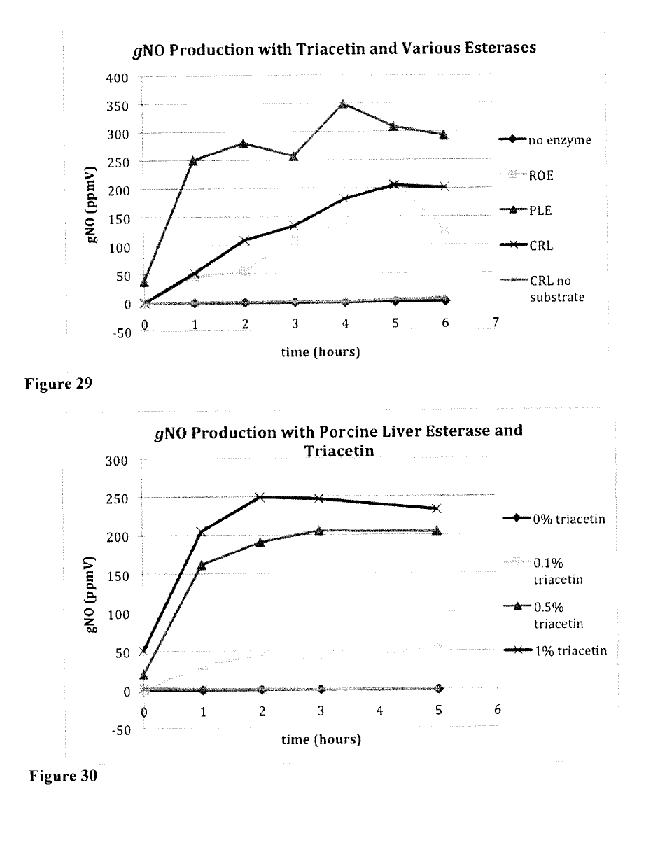

FIG. 29 presents an experiment testing three different esterase or lipase against triacetin. The comparison shows that porcine liver esterase reaches above 200 ppmV within an hour while the lipases take slightly more time. Both candida rugosa lipase and rhyzopus oryzae esterase also reach 200 ppmV but in 4-5 hours. It is important to note however that the concentration of enzyme will affect the time required to reach the maximum production of nitric oxide as well as the duration of production. Another element altering the level of nitric oxide produced by the enzymes is the substrate concentration of the assay. Varying the concentration of triacetin controls the production of nitric oxide (FIG. 30). The production can reach up to 250 ppmV using 1% triacetin in the assay while the use of 0.5% will limit the production to 200 ppmV. This interplay between enzyme and substrate allows for a fine adjustment of the level of production, an important aspect for the creation of wound healing dressings or dressings for treating a microbial infection or dermatological disorder.

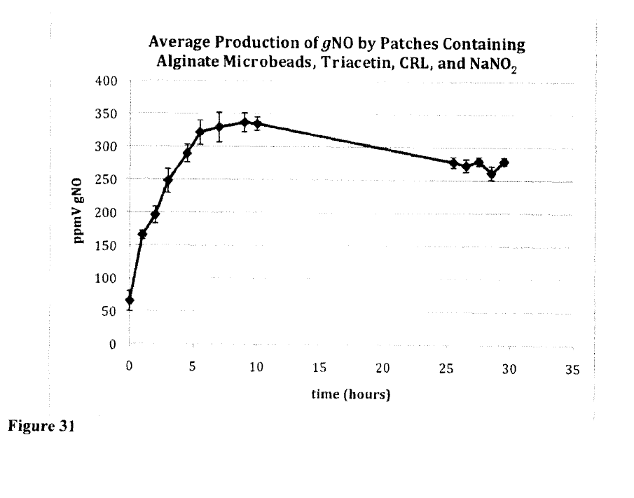

The enzymatic production of gNO was tested in dressings composed of Tegaderm (3M) non-occlusive dressings, polyethylene membrane and a gas impermeable upper layer of aluminium adhesive. The dressings were based on the use of candida rugosa lipase as the esterase and triacetin as the triglyceride substrate. FIG. 31 shows that production of nitric oxide rapidly reached the goal of 200 ppmV and was maintained at a biologically active level above 200 ppmV for 30 hours. This formulation can be used for the production of dressings for the treatment of chronic wounds, microbial infections or dermatological disorders.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
SEQUENCE LIST

SEQ. ID. NO. 1
LOCUS YP_001271831, 375 aa, linear, BCT 06-DEC-2007
DEFINITION Nitric-oxide synthase [Lactobacillus reuteri F275].
SOURCE Lactobacillus reuteri F275
ORIGIN

| | |
|---|---|
| 1 | mteqeqqtee lrcigcgsii qtedpnglgy tpksalekgk etgelycqrc frlrhyneia |
| 61 | pvsltdddfl rllnqirdan alivyvvdvf dfngslipgl hrfvgdnpvl lvgnkedllp |
| 121 | rslrrpkltd wirqqaniag lrpidtvlvs akknhqidhl ldviekyrhn rdvyvvgvtn |
| 181 | vgkstlinqi ikqrtgvkel ittsrfpgtt ldkieipldd ghvlvdtpgi ihqeqmahv1 |
| 241 | spkdlkivap qkeikpktyq lndgqtlflg gvarfdylhg eragmvayfd nnlpihrtkl |
| 301 | nnadnfyakh lgdlltppts deknefpple ryefhiteks divfeglgwi tvpakttvaa |
| 361 | wvpkgvgalv rrami |

SEQ. ID. NO. 2
LOCUS ZP_01273963, 1221 aa, linear, BCT 14-APR-2006
DEFINITION Nitrate reductase, alpha subunit [Lactobacillus reuteri
100-23].
SOURCE Lactobacillus reuteri 100-23
ORIGIN

| | |
|---|---|
| 1 | mksrffnkvd kfngtftqle ensrrwekly rqrwandkvv rtthgvnctg scswnvyvkq |
| 61 | giitwehqat dypscgpnip gyeprgcprg asfswyeysp vrikypyirg klwelwtaak |
| 121 | kehenpldaw asivedpeks kkykkvrghg glirvhryea lemisaacly tikkygpdri |
| 181 | ggftpipams mmsfsagarf ialmggeqms fydwyadlpp aspqvwgeqt dvpesaewyn |
| 241 | ssyiimwgsn vpltrtpdah fmtevrykgt kivayspdya envkfaddwl apepgsdsav |
| 301 | aqamtyvild efyqkhpvkr fidyskrftd 1pfmveleps tanddhytpg rfvrisdlvd |
| 361 | ddtivnpawk tvvydqnnhk ivvpngtmgq eynvkekwnl elldqngnki dpalsindqg |
| 421 | geteqiiadf pafsndgnsv vqrhlpvkkl kftdgqehlv tnvydlmmaq mgidrtgndd |
| 481 | laakdamdae syftpawqes rsgvkaeqvi qiarefaqna aenegrsmvi mgggvnhwfn |
| 541 | admnyrniin mlmlcgcvgm tgggwahyvg qeklrpqegw anitfandwe kggarqmqgt |
| 601 | twyyfatdqw ryeeidnqaq kspvwkskhs ylhnadynqm airlgwlpsy pqfdrnplsf |
| 661 | akdynttdid eiskkvvdel kkgtlhfaae dpdanqnqpk afflwrsnlf assgkgaeyf |
| 721 | mkhllgaeng llakpndrvk pqdmiwrdkg avgkldlvvd mdfrmvstpm ysdvvlpaat |
| 781 | wyekkdlsst dmhpfihpfn aaispmwesk sdwqqfklla ktisemakky mpgtfydlks |
| 841 | aplghntqge iaqpygkikd wkngetepip gktmpslklv trdytkiydk fitlgpnivn |
| 901 | nygynvaaqy dylkgmngta segigagcpl ldedekvcda ilrmstasng kladrawekk |
| 961 | qertgehltd igrghaddsm sfkqitaqpq eayptpigts akhggarytp fslmternip |
| 1021 | tftltgrqhf yidheifref genmatykps lppvvmapgd vdvppvkdev tlkymtphgk |
| 1081 | wnihtmyydn lemltlfrgg ptiwispqda dkikvkdndw ievynrngvv taravvsvrm |
| 1141 | pegsmymyha qdneiyepls titgnrggsh naptqihvkp thmvggygql sygwnyygpt |
| 1201 | gnqrdlyanv rklrkvnwse d |

SEQ. ID. NO. 3
LOCUS ZP_01273962, 519 aa, linear, BCT 14-APR-2006
DEFINITION Nitrate reductase, beta subunit [Lactobacillus reuteri
100-23].
SOURCE Lactobacillus reuteri 100-23
ORIGIN

| | |
|---|---|
| 1 | mkikaqismv lnldkcigch tcsvtckntw tnrpgaeymw fnnvetkpgv gypkrweded |
| 61 | qyhggwtlns kgklklrags klnkialgki fynndmpeld nyyepwtydy ktlfgpeqkh |
| 121 | qpvarpksqi tgegmelttg pnwdddlags teyvqqdpnm qkiegdiknn feqafmmylp |
| 181 | rlcehclnap cvascpsgam ykrdedgivl vdqercrgwr fcmtgcpykk vyfnwkthka |
| 241 | ekctfcypri eegqptvcae tcvgriryig ailydadrve eaastpdesk lyeaqlglfl |
| 301 | dpndpevvkq alkdgiseem ieaaqkspiy kmavkekiaf plhpeyrtmp mvwyipplsp |
| 361 | vmsyfegrds iknpemifpg idqmrvpvqy laslltagnv pvikkalykl ammrlymrak |
| 421 | tsgrdfdssk lervdlteer atslyrllai akyedrfvip ssqkaemeda qteqqslgyd |
| 481 | ecegcalapq hksmfkkaea gkstnqiyad sfyggiwrd |

SEQ. ID. NO. 4
LOCUS ZP_01273960, 229 aa, linear, BCT 14-APR-2006
DEFINITION Nitrate reductase, gamma subunit [Lactobacillus reuteri
100-23].
SOURCE Lactobacillus reuteri 100-23
ORIGIN

| | |
|---|---|
| 1 | mhngwsiflw viypyimlas ffigtfvrfk yfhpsitaks selfekkwlm igsitfhigi |
| 61 | ilaffghclg mfipaswtay fgitehmyhi fgslmmgipa gilafvgiai ltyrrmtcsr |
| 121 | vyktsdindi ivdwallvti alglactitg afidynyrtt ispwarslfv lnpqwqlmrs |
| 181 | vpliykihvl cglaifgyfp ytrlvhaltl pwqyifrrfi vyrrrarvy |

SEQ. ID. NO. 5
LOCUS ZP_01273961, 192 aa, linear, BCT 14-APR-2006
DEFINITION Nitrate reductase, delta subunit [Lactobacillus reuteri
100-23].
SOURCE Lactobacillus reuteri 100-23
ORIGIN

| | |
|---|---|
| 1 | midfrrltdl kdtfavlsrl idypdtetfs peirqllltd nalstatrge llslfdelaa |
| 61 | lssievqemy ahlfemnrry tlymsyykmt dsrergtila rlkmlyemfg iseatselsd |
| 121 | ylplllefla ygdytndprr qdiqlalsvi edgtytllkn avtdsdnpyi qlirltrsli |
| 181 | gscikmevre da |

TABLE 2
Nitric oxide (NO) biosynthesis from arginine by nitric oxide
synthase (NOS) in the presence of oxygen and NADPH

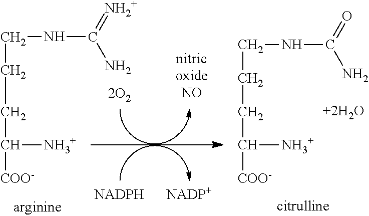

TABLE 3
Nitric oxide (NO) production by reduction of nitrite (NO₂) salts

NO₂ + 2H + → H₂O + NO

TABLE 4
nitric oxide (NO) production by reduction of nitrate (NO₃)salts
to nitrite (NO₂) and then reduction of NO₂ to nitric oxide gas gNO

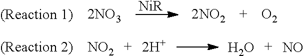

TABLE 5
HISTOLOGICAL WOUND EVALUATION FOR ISCHEMIC WOUNDS

| | | Control | Treatment | trend |
|---|---|---|---|---|
| Wound surface | Wound width (% initial) | 1.00 ± 0.27 | 1.08 ± 0.19 | |
| | Raised (+)/depressed (−) (0 to 3) | −1.00 ± 1.77 | 0.14 ± 1.68 | Improved |
| | Central protrusion | 0.13 ± 0.35 | 0.57 ± 0.98 | |
| | Crusting/exudates (0 to 3) | 1.63 ± 1.41 | 0.5 ± 1.07 | Improved |
| Epidermis | Cover (%) | 79.4 ± 29.8 | 87.5 ± 31.5 | Improved |
| | Hyperplasia (0 to 3) | 2.63 ± 0.74 | 2.29 ± 0.76 | Improved |
| | Maturity (1 to 4) | 2.38 ± 0.91 | 3.13 ± 0.64 | Improved |
| Granulation tissue/dermis | Thickness | 0.84 ± 0.76 | 1.13 ± 0.64 | improved |
| | Inflammation/infiltration | 2.38 ± 0.74 | 2.13 ± 0.83 | improved |
| | maturity | 1.13 ± 0.83 | 1.88 ± 0.99 | improved |

TABLE 6

| | | | vehicle | | treated | | |
|---|---|---|---|---|---|---|---|
| | | | rabbit 1 | rabbit 3 | rabbit 2 | rabbit 4 | Finding |
| Weight loss | | | 0.1 kg | 0.3 kg | 0.1 kg | 0 kg | No difference |
| blood morphology | | | Normal | Normal | Normal | Normal | No difference |
| Hematology | WBC | (×10⁸/L) | 6.16 | 1.54 | 7.81 | 4.66 | |
| | RBC | (×10¹²/L) | 5.91 | 6.10 | 6.07 | 5.98 | |
| | HGB | (g/L) | 122 | 121 | 119 | 122 | |
| | HCT | (L/L) | 0.35 | 0.34 | 0.35 | 0.36 | |
| | MCV | (fL) | 59.9 | 55.2 | 57.7 | 60.3 | Normal profiles |
| | MCH | (pg) | 20.6 | 19.8 | 19.7 | 20.5 | |
| | MCHC | (g/L) | 344 | 359 | 341 | 339 | (Low WBC in rabbit |
| | PLT | (×10⁹/L) | 315 | 444 | 247 | 583 | #3, untreated animal, |
| | Neut | (×10⁹/L) | 1.85 | 0.38 | 1.52 | 1.32 | unrelated to |
| | Lymp | (×10⁹/L) | 3.69 | 1.07 | 5.75 | 2.98 | NO treatment) |
| | Mono | (×10⁹/L) | 0.06 | 0.01 | 0.07 | 0.05 | |
| | Eos | (×10⁹/L) | 0.11 | 0.03 | 0.15 | 0.09 | |
| | Luc | (×10⁹/L) | 0.01 | 0.00 | 0.01 | 0.00 | |
| | Baso | (×10⁹/L) | 0.43 | 0.05 | 0.31 | 0.22 | |
| | Retic | (×10^{12/}L) | 0.211 | 0.086 | 0.163 | 0.137 | |
| Blood chemistry | Chol | mmole/l | 0.65 | 0.92 | 0.59 | 0.62 | |
| | TG | mmole/l | 0.97 | 0.96 | 1.12 | 0.98 | |
| | ALT | U/l | 50.83 | 31.01 | 28.71 | 32.12 | |
| | AST | U/l | 32.89 | 33.3 | 54.95 | 19.32 | |
| | Crea | μmoles/l | 126.84 | 162.61 | 144.78 | 127.26 | Normal profiles |
| | HDL-c | mmole/l | 0.3 | 0.41 | 0.08 | 0.24 | |
| | urea | mmole/l | 6.99 | 6.17 | 7.15 | 6.57 | (High K in rabbits |
| | lip | U/l | 190.59 | 158.6 | 148.37 | 194.28 | #3 and #4, |
| | glu | mmole/l | 13.73 | 11.22 | 12.56 | 14.74 | Unrelated to |
| | CA | mmole/l | 3.29 | 3 | 3.26 | 3.16 | Treatment) |
| | Phos | mmole/l | 2.11 | 2.06 | 2.74 | 1.97 | |
| | Co2-L | mmole/l | 34.13 | 30.86 | 27.52 | 22.76 | |
| | CRP-s | nmole/l | 0.14 | 1.88 | −0.38 | 1.34 | |
| | Na | mmole/l | 146.8 | 146.3 | 148 | 148 | |
| | k | mmole/l | 5.79 | 9.79 | 5.55 | 11.44 | |
| | Cl | mmole/l | 101.5 | 102.9 | 107.5 | 109.2 | |

| | | | |
|---|---|---|---|
| Methemoglobine | 0.3% ± 0.2% | 0.1% ± 0.1% | Normal levels |

TABLE 7
(corresponds to FIG. 25)
Cox Proportional Hazards

| | Hazard | | | | | Z- | P- |
|---|---|---|---|---|---|---|---|
| Term | Ratio | 95% | C.I. | Coefficient | S.E. | Statistic | Value |
| treated (Yes/No) | 2.5166 | 1.0548 | 6.0043 | 0.9229 | 0.4437 | 2.0801 | 0.0375 |

| | |
|---|---|
| Convergence: | Converged |
| Iterations: | 4 |
| −2 * Log-Likelihood: | 131.1777 |

| Test | Statistic | D.F. | P-Value |
|---|---|---|---|
| Score | 4.6006 | 1 | 0.032 |
| Likelihood Ratio | 4.4189 | 1 | 0.0355 |

TABLE 8
(corresponds to FIG. 26)

| Test | Statistic | D.F. | P-Value |
|---|---|---|---|
| Log-Rank | 5.2097 | 1 | 0.0225 |
| Wilcoxon | 6.4173 | 1 | 0.0113 |

### REFERENCES

Adams L B, Hibbs J B Jr., Taintor R R, Krahenbuhl J L, Microbiostatic effect of murine-activated macrophages for Toxoplasma gondii, J. Immunol. 1990, 144, 2725-2729
Anstead, G. M., Quinones-Nazario, G., & Lewis, J. S. Treatment of infections caused by resistant Staphylococcus aureus. Methods Mol. Biol. 391, 227-258 (2007).
Anstey N M, Weinberg J B, Hassanali M Y, Mwaikambo E D, Manyenga D, Misukonis M A, Arnelle D R, Hollis D, McDonald M I, Granger D L, Nitric oxide in Tanzanian children with Malaria. Inverse relationship between malaria severity and nitric oxide production/nitric oxide synthase type 2 expression, J. Exp. Med. 1996, 184, 557-567
Archer, S. Measurement of Nitric Oxide in Biological Systems. The FASEB Journal 7, 349-360 (1993).
Augusto O, Linares E, Giorgio S, Possible roles of nitric oxide and peroxynitrite in murine leishmaniasis, Braz. J. Med. Biol. Res. 1996, Jul. 29, 7, 853-862
Blackytny, R. & Jude, E. The molecular biolgy of chronic wounds and delayed healing in diabetes. Diabetic Medicine 23, 594-608 (2006).
Cals-Grierson, M. M. & Ormerod, A. D. Nitric oxide function in the skin. Nitric. Oxide. 10, 179-193 (2004).
DeGroote M A and Fang F C, NO inhibitions: antimicrobial properties of Nitric Oxide. Clin. Infect. Dis. 1995, 21 (Suppl. 2), S162-S165
DeGroote M A, Granger D, Xu Y, Campbell G, Prince R, Fang F C, Genetic and redox determinants of nitric oxide cytotoxicity in a Salmonella typhimurium model. Proc. Natl. Acad. Sci. USA, 1995, 92, 6399-6403
Evans T G, That L, Granger D L, Hibbs J B Jr., Effect of in vivo inhibition of nitric oxide production in murine leishmaniasis, J. Immunol. 1993, 151, 907-915
Fang F C, Perspective series: host/pathogen interactions. Mechanisms of nitric oxide-related antimicrobial activity, J. Clin. Invest. 99 (1997) 2818-2825.
Ghaffari et al. Potential application of gaseous nitric oxide as a topical antimicrobial agent: Nitric Oxide Biology and Chemistry. 14 (2006) 21-29.
Gorwitz, R. J. A review of community-associated methicillin-resistant Staphylococcus aureus skin and soft tissue infections. Pediatr. Infect. Dis. J. 27, 1-7 (2008).
Gracie, J. A. et al. A proinflammatory role for IL-18 in rheumatoid arthritis. J. Clin. Invest 104, 1393-1401 (1999).
Hegesh, E., Gruener, N., Cohen, S., Bochkovsky, R., & Shuval, H. I. A sensitive micromethod for the determination of methemoglobin in blood. Clin. Chim. Acta 30, 679-682 (1970).
Ischiropoulos H, Al-Mehdi A B, Peroxynitrite-mediated oxidative protein modifications. FEBS 1995, 364, 279-282
Juedes M J, Wogan G N, Peroxynitrite-induced mutation spectra of pSP189 following replication in bacteria and in human cells, Mutat. Res. 1996, 349, 51-61
Kawanishi M, Nitric Oxide inhibits Epstein-Barr virus DNA replication and activation of latent EBV, Intervirology 1995, 38 (3-4), 206-213
Kikuchi, K., Nagano, T. and Hirobe, M. Novel detection method of nitric oxide using horseradish peroxidase. Biol. Pharm. Bull. 1996 April; 19(4):649-651.
Linares, J. The VISA/GISA problem: therapeutic implications. Clin. Microbiol. Infect. 7 Suppl 4, 8-15 (2001).
McInnes, I. B. & Liew, F. Y. Immunomodulatory Effects of Nitric Oxide in Nitric Oxide and Infection 199-213 (Springer US, 1999).
Michelakis, E. D. & Archer, S. L. The measurement of NO in biological systems using chemiluminescence. Methods Mol. Biol. 100, 111-127 (1998).
Mollace, V., Salvemini, D., Anggard, E., & Vane, J. Cultured astrocytoma cells inhibit platelet aggregation by releasing a nitric oxide-like factor. Biochem. Biophys. Res. Commun. 172, 564-569 (1990).
Morris S L, Hansen J N, Inhibition of Bacillus cereus spore outgrowth by covalent modification of a sulfhydryl group by nitrosothiol and iodoacetate, J Bacteriol, 1981, 148, 465-471
Mulligan, M. S., Hevel, J. M., Marletta, M. A., & Ward, P. A. Tissue injury caused by deposition of immune complexes is L-arginine dependent. Proc. Natl. Acad. Sci. U.S. A 88, 6338-6342 (1991).
Nordmann, P., Naas, T., Fortineau, N., & Poirel, L. Superbugs in the coming new decade; multidrug resistance and prospects for treatment of Staphylococcus aureus, Enterococcus spp. and Pseudomonas aeruginosa in 2010. Curr. Opin. Microbiol. 10, 436-440 (2007).
Palmer, R. M., Ashton, D. S., & Moncada, S. Vascular endothelial cells synthesize nitric oxide from L-arginine. Nature 333, 664-666 (1988).
Serarslan, G., Altug, E., Kontas, T., Atik, E., & Avci, G. Caffeic acid phenethyl ester accelerates cutaneous wound healing in a rat model and decreases oxidative stress. Clin. Exp. Dermatol. 32, 709-715 (2007).
Stenzler, A. & Miller, C. Device and method for treatment of wounds with nitric oxide. Ser. No. 11/021,109 [U.S. Pat. No. 7,122,018], 1-21. 2006. United States. Ref Type: Patent
Vasquez-Torres A, Jones-Carson J, Balish E, Peroxynitrite contributes to the candidacidal activity of nitric oxide-producing macrophages, Infect. Immun. 1996, 64, 3127-3133
Wolf, G., Arendt, E. K., Pfahler, U., & Hammes, W. P. Heme-dependent and heme-independent nitrite reduction by lactic acid bacteria results in different N-containing products. Int. J. Food Microbiol. 10, 323-329 (1990).
Xu, J. & Verstraete, W. Evaluation of nitric oxide production by lactobacilli. Appl. Microbiol. Biotechnol. 56, 504-507 (2001).
Zeina B, Banfield C, al-Assad S, Topical glyceryl trinitrate: a possible treatment for cutaneous leishmaniasis, Clin. Exp. Dermatol. 1997 22, 5, 244-245

1. A composition comprising a) an isolated enzyme or a live cell expressing an endogenous enzyme, the enzyme (i) having activity that converts a nitric oxide gas precursor to nitric oxide gas or (ii) having activity on a substrate that produces a catalyst that causes the conversion of the nitric oxide gas precursor to nitric oxide gas, or b) a live cell producing a catalyst for converting the nitric oxide gas precursor to nitric oxide gas; and a carrier.

2. The composition of claim 1, further comprising the nitric oxide gas precursor.

3. (canceled)

4. The composition of claim 1, wherein the carrier comprises a matrix selected from a natural polymer, a synthetic polymer, a hydrogel, a natural gel, dissolvable film, multi-part or layered dissolvable film, a microcapsule, liposome, hydrocarbon-based and petroleum jelly.

5.-10. (canceled)

11. The composition of claim 1, wherein the composition is a cream, slab, gel, hydrogel, dissolvable film, spray, paste, emulsion, patch, liposome, balm or mask.

12. A device for delivering nitric oxide gas to affected tissue comprising a casing comprising a barrier surface and a contact surface, said contact surface being permeable to nitric oxide gas, wherein the casing contains the composition of claim 1, the composition located between the barrier surface and the contact surface.

13. (canceled)

14. The device of claim 12, wherein the casing separates the composition from the tissue and the casing is impermeable to the composition.

15. The composition of claim 1, wherein the affected tissue comprises wounded skin, microbially infected skin and/or skin affected by a dermatological disorder and the composition is suitable for topical administration to the skin.

16.-22. (canceled)

23. The composition of claim 1, wherein the enzyme is a nitrate reductase (NaR), nitrite reductase (NiR), nitric oxide synthase (NOS), glutathione S-transferase (GST), or cytochrome P450 system (P450).

24. The composition of claim 1, wherein the catalyst comprises protons and wherein the protons are a product or by-product of the enzyme reaction.

25. (canceled)

26. (canceled)

27. The composition of claim 1, wherein the enzyme and substrate comprise lipase and triglyceride, esterase and ester, lipase and ester, esterase and triglyceride, protease and protein, trypsin and protein, chymotrypsin and protein.

28. The composition of claim 1 wherein the enzyme is lipase and the substrate is triacetin.

29. (canceled)

30. (canceled)

31. The composition of claim 1, wherein a reducing agent and/or an enzyme cofactor is added.

32. (canceled)

33. The device of claim 12, wherein the barrier surface is impermeable to oxygen.

34.-40. (canceled)

41. The composition of claim 1, wherein the cell is a probiotic microorganism of the genus Lactobacillus, Bifidobacteria, Pediococcus, Streptococcus, Enterococcus, or Leuconostoc.

42.-46. (canceled)

47. The composition of claim 1, wherein the cell is microencapsulated.

48.-50. (canceled)

51. The composition of claim 1, wherein the cell or enzyme or precursor is immobilized in a reservoir.

52. (canceled)

53. (canceled)

54. The composition of claim 1, wherein the composition further comprises growth media for cells such as MRS broth, LB broth, glucose, or other carbon source containing growth media.

55. (canceled)

56. The composition of claim 1, wherein i) the enzyme comprises nitrite reductase (NiR) and the nitric oxide gas precursor comprises a nitrite or salt thereof, ii) the enzyme comprises nitric oxide synthase (NOS), and the nitric oxide precursor comprises L-arginine, or iii) the enzyme comprises nitrate reductase (NaR) and the nitric oxide gas precursor is a nitrate or salt thereof.

57.-65. (canceled)

66. The device of claim 12, further comprising a nitric oxide gas concentrating substance comprising a spacer, a gas cell containing structure, or a sponge for collection of the nitric oxide gas.

67.-70. (canceled)

71. The device of claim 12, wherein the casing comprises a plurality of layers, wherein the layers comprise:
a) a barrier layer;
b) a contact layer; and
c) an active layer.

72. (canceled)

73. The device of claim 71, further comprising a reservoir layer.

74. (canceled)

75. (canceled)

76. The device of claim 73, further comprising at least one valve connecting the active layer and the reservoir layer, wherein the valve has an initial closed position in which the cell or enzyme are separate from the precursor and an open position in which the active layer and reservoir layer are in fluid communication, and the cell or enzyme or precursor are permitted to flow between the layers.

77. The device of claim 76, wherein the valve comprises a one-way valve, and wherein in the open position either the enzyme or cell or the precursor is permitted to flow between the layers or wherein the valve comprises a pressure actuated valve that is actuable from the closed position to the open position by compression of the device.

78.-80. (canceled)

81. The device of claim 73, wherein the nitric oxide (NO) is produced in a chemical reaction between an acid produced by a lactic acid producing bacteria (LAB) in the active layer and an NO containing substrate in the reservoir layer.

82. The composition of claim 1, wherein the composition has an inactive composition state and an active composition state, wherein in the inactive composition state, the composition is dehydrated and the precursor does not interact with the enzyme or catalyst to produce NO gas and wherein in the active composition state, the composition is hydrated and the precursor is converted to NO gas by the enzyme or catalyst.

83. (canceled)

84. (canceled)

85. A method for treating a wound, a microbial infection and/or a dermatological disorder in a subject in need thereof comprising:
(a) contacting affected tissue with (i) a nitric oxide gas releasing composition, the composition containing a plurality of inactive agents that, upon activation, react to produce nitric oxide gas or (ii) a device comprising a casing permeable to nitric oxide gas, the casing containing a plurality of inactive agents that, when activated, react to produce nitric oxide gas;
(b) activating the inactive agents to produce nitric oxide gas, wherein the nitric oxide gas contacts the affected tissue for treating the wound, microbial infection and/or dermatological disorder in the subject in need thereof; and wherein the inactive agents comprise i) a nitric oxide gas precursor, ii) (a) an isolated enzyme or a live cell expressing an endogenous enzyme, the enzyme 1) having activity that converts the nitric oxide gas precursor to nitric oxide gas or 2) having activity on a substrate that produces a catalyst that causes the conversion of the nitric oxide gas precursor to nitric oxide gas or (b) a live cell producing a catalyst for converting nitric oxide gas precursor to nitric oxide gas; and a carrier.

86. (canceled)

87. The method of claim 85, wherein the inactive agents comprise separated agents and activating the separated agents comprises combining the separated agents.

88. The method of claim 87, wherein the separated agents are activated in step (b) by mixing the separated agents by applying pressure or temperature to the device or composition.

89. The method of claim 85, wherein the inactive agents are dehydrated agents and activating the inactive agents comprises hydrating the agents.

90. A method for treating a wound, a microbial infection and/or a dermatological disorder in a subject in need thereof comprising:
contacting tissue with a nitric oxide gas releasing composition or device, the composition or device comprising an isolated enzyme or a live cell expressing an endogenous enzyme, the enzyme (i) having activity that converts nitrate to nitric oxide gas or (ii) having activity on a substrate that produces a catalyst that causes the conversion of nitrate to nitric oxide gas or (b) a live cell expressing a catalyst for converting nitrate to nitric oxide gas;
wherein the composition reacts with nitrate in sweat on the tissue to produce nitric oxide gas for treating a wound, a microbial infection and/or a dermatological disorder in the subject in need thereof.

91. (canceled)

92. A method for treatment of a wound, a microbial infection and/or a dermatological disorder in a subject in need thereof comprising exposing affected tissue to the composition of claim 1, wherein NO produced by the composition contacts the affected tissue.

93.-114. (canceled)

115. The method of claim 85, wherein the subject is a human.

116.-118. (canceled)
